SEQUENCE LISTING (1 sequence)

SEQ ID NO: 1 - protein, 35 aa, Artificial sequence
  Synthetic peptide
  MOD_RES (20)..(20): X = MMT-Lys
FNKEQQNAFYEILHLPNLNXEQRNAFIQSLKDDPS

(19) United States
(12) Patent Application Publication
(10) Pub. No.: US 2024/0299568 A1
(43) Pub. Date: Sep. 12, 2024

(54) BONE-SPECIFIC DELIVERY OF POLYPEPTIDES

(71) Applicants: William Marsh Rice University, Houston, TX (US); Baylor College of Medicine, Houston, TX (US)

(72) Inventors: Han XIAO, Houston, TX (US); Xiang ZHANG, Houston, TX (US)

(73) Assignees: William Marsh Rice University, Houston, TX (US); Baylor College of Medicine, Houston, TX (US)

(21) Appl. No.: 18/261,983

(22) PCT Filed: Jan. 19, 2022
(86) PCT No.: PCT/US2022/012982

§ 371 Date: Jul. 18, 2023

Related U.S. Application Data
(60) Provisional application No. 63/138,972, filed on Jan. 19, 2021.

(51) Int. Cl. A61K 47/68; A61K 47/64; A61P 35/04

(52) U.S. Cl. CPC A61K 47/6803; A61K 47/64; A61K 47/6851; A61P 35/04

(57) ABSTRACT

The present disclosure provides methods for treating bone diseases, such as bone cancers, bone metastasis of cancers, or osteoporosis, by administering a bone-targeting polypeptide conjugate. The bone-targeting polypeptide conjugate may be a bone-targeting antibody conjugate.

### PRIORITY CLAIM

This application claims benefit of priority to U.S. Provisional Application Ser. No. 63/138,972, filed Jan. 19, 2021, the entire contents of which are hereby incorporated by reference.

### STATEMENT REGARDING FEDERALLY SPONSORED RESEARCH OR DEVELOPMENT

The invention was made with government support under grants W81XWH-16-1-0073 and W81XWH-21-1-0789 awarded by the Department of Defense. The government has certain rights in the invention.

### BACKGROUND

### 1. Field

The disclosure relates generally to the field of molecular biology. More particularly, it concerns methods of site-specific delivery of an antibody.

### 2. Related Art

Antibody-based therapies, including those using monoclonal antibodies, antibody-drug conjugates, bispecific antibodies, checkpoint inhibitors, and others, have realized their clinical potential in terms of their power to treat a variety of cancers.¹⁻⁴ Nevertheless, despite the fact that most therapeutic antibodies have high affinities for their targets, the presence of these same targets in normal tissues can dramatically limit the ability of therapeutic agents to hit their targets without inducing unacceptable “on-target” toxicity in healthy cells.⁵⁻⁷ Furthermore, low levels of delivery of therapeutic antibodies to some tissues such as brain or bone can significantly limit their efficacy in treating diseases in these tissues.⁸ Thus, it is likely that enhancing both the antigen and tissue specificity of antibodies will ultimately transform the efficacy of antibody therapy for clinical treatment of cancer.

Half of patients with an initial diagnosis of metastatic breast cancer (BCa) will develop bone metastases.⁹ Patients having only skeletal metastases usually have a better prognosis than patients with vital organ metastases.^{9,10} Furthermore, bone metastasis is associated with severe symptoms such as spinal cord compression, pathological fractures, and hypercalcemia.” Despite the deep understanding of molecular mechanisms,^{12,13} effective therapies that can eliminate cancer cells are still lacking.” Bone is not the final destination of metastatic dissemination. Recent genomic analyses have revealed frequent “metastasis-to-metastasis” seeding.¹⁵⁻¹⁷ Over two-thirds of bone-only metastases subsequently develop secondary metastases to other organs, ultimately leading to the death of patients.^{9,10} In fact, some metastases initially identified in non-bone organs are actually the result of seeding from sub-clinical bone micrometastases (BMMs). This apparently is the result of cancer cells initially arriving in the bone and then acquiring more aggressive phenotypes that allow them to establish more overt metastases in both bone and other sites.¹⁸ Thus, there is an unmet need for strategies for preventing BMMs from establishing more overt metastases in both bone and non-bone tissues.

While targeted antibody therapy and immunotherapy are currently emerging as new avenues for treating metastatic breast cancer, the performance of these agents in patients with bone metastases has been disappointing. For example, trastuzumab (Herceptin) and pertuzumab (Perjeta) antibodies targeting human epidermal growth factor receptor 2 (HER2) have been used to treat patients in adjuvant and metastatic settings. Although many BCa patients benefit from these treatments, in large numbers of BCa patients with bone metastasis, the disease progresses within one year and few patients experience prolonged remission.¹⁹⁻²² In another phase III clinical trial testing atezolizumab in patients with metastatic triple-negative BCa, progression-free survival was significantly longer in the atezolizumab group than in the placebo group. However, among BCa patients with bone metastases, no significant difference was observed between the atezolizumab-treated and placebo groups for risk of progression or death.²³ Therapies with improved outcomes for BCa patients with bone metastases are therefore highly desired.

### SUMMARY

In certain embodiments, the present disclosure provides methods for treating or preventing bone diseases in a subject comprising administering to the subject an effective amount of a bone-targeting conjugate comprising bisphosphonate (BP) conjugated to a polypeptide or protein. In one embodiment, the present disclosure provides methods for treating or preventing bone tumors in a subject comprising administering to the subject an effective amount of a bone-targeting conjugate comprising bisphosphonate (BP) conjugated to an antibody.

In certain aspects, the bone disease is osteoporosis, osteomalacia, periodontitis, rheumatoid arthritis, metabolic bone disease, a parathyroid disorder, steroid-induced osteoporosis, chemotherapy-induced bone loss, pre-menopausal bone loss, fragility and recurrent fractures, renal osteodystrophy, bone infections, or Paget's disease. The methods and compositions provided herein may be used to reduce cortical and/or trabecular bone loss, reduce cortical and/or trabecular bone mineral content loss, improve the bone biomechanical resistance, increase bone formation, and/or reduce bone-resorption.

In some aspects, the subject has bone cancer or bone metastasis. In particular aspects, the bone cancer is Ewing sarcoma, osteosarcoma, or chondrosarcoma. In certain aspects, the bone metastasis is from breast cancer, myeloma, renal cancer, lung cancer, prostate cancer, thyroid cancer, or bladder cancer. In specific aspects, the bone metastasis is breast cancer bone metastasis. In some aspects, the breast cancer is triple-negative breast cancer. In certain aspects, the breast cancer is HER2-negative breast cancer. In other aspects, the breast cancer is HER2-positive breast cancer.

In certain aspects, the BP is negatively-charged. In some aspects, the BP is alendronate, zoledronate, pamidronate, risedronate, medronic acid, aminomethylene bisphonic acid, clodronate, etidronate, tiludronate, ibandronate pomidronate, neridonate, olpadronate, or oxidronate. In particular aspects, the BP is alendronate (ALN).

In some aspects, the BP is conjugated to an adrenergic agonist, an anti-apoptosis factor, an apoptosis inhibitor, a cytokine receptor, a cytokine, a cytotoxin, an erythropoietic agent, a glutamic acid decarboxylase, a glycoprotein, a growth factor, a growth factor receptor, a hormone, a hormone receptor, an interferon, an interleukin, an interleukin receptor, a kinase, a kinase inhibitor, a nerve growth factor, a netrin, a neuroactive peptide, a neuroactive peptide receptor, a neurogenic factor, a neurogenic factor receptor, a neuropilin, a neurotrophic factor, a neurotrophin, a neurotrophin receptor, an N-methyl-D-aspartate antagonist, a plexin, a protease, a protease inhibitor, a protein decarboxylase, a protein kinase, a protein kinase inhibitor, a proteolytic protein, a proteolytic protein inhibitor, a semaphorin, a semaphorin receptor, a serotonin transport protein, a serotonin uptake inhibitor, a serotonin receptor, a serpin, a serpin receptor, or a tumor suppressor.

In some aspects, the antibody is a monoclonal antibody, bispecific antibody, Fab′, a F(ab′)2, a F(ab′)3, a monovalent scFv, a bivalent scFv, a single domain antibody, or nanobody. In certain aspects, the antibody is an immune checkpoint inhibitor. In particular aspects, the antibody is an anti-HER2 antibody, anti-CD99 antibody, anti-IGF-IR antibody, anti-PD-L1, anti-PD-1, anti-CTLA4-antibody, anti-Siglec-15 antibody, anti-RANKL antibody, or anti-TGFβ antibody. In specific aspects, the antibody is an anti-HER2 antibody, such as trastuzumab (Herceptin), pertuzumab (Perjeta), or atezolizumab. In particular aspects, the antibody is trastuzumab. In some aspects, bone-targeting conjugate comprises alendronate conjugated to trastuzumab. In certain aspects, the antibody is not an anti-M-CSF antibody.

In certain aspects, the BP is not conjugated to N-glycan on the Fc region of the antibody. In some aspects, the BP is site-specifically conjugated to the antibody using pClick conjugation, NHS-ester chemistry, or cysteine chemistry. In particular aspects, the BP is site-specifically conjugated to the antibody using pClick conjugation. In some aspects, the BP is conjugated to the CH2-CH3 junction of the antibody. In some aspects, the BP is conjugated to the antibody using 4-fluorophenyl carbamate lysine (FPheK). In particular aspects, FPheK is attached to a fragment of the B domain of protein A (FB protein) from Staphylococcus aureus. In specific aspects, pClick conjugation comprises conjugation of an antibody with an azide functional moiety with BP functionalized with bicyclo[6.1.0]nonyne (BCN).

In certain aspects, the bone-targeting conjugate results in increased concentration of therapeutic antibody at the bone tumor niche, inhibits cancer development in the bone, and/or limits secondary metastases to other organs. In some aspects, the bone-targeting conjugate results in decreased micrometastasis-induced osteolyic lesions.

In additional aspects, the method comprises further administering an additional anti-cancer therapy. In some aspects, the additional anti-cancer therapy comprises surgery, chemotherapy, radiation therapy, hormonal therapy, immunotherapy or cytokine therapy. In particular aspects, the additional anti-cancer therapy comprises immunotherapy or chemotherapy.

A further embodiment provides the use of a bone-targeting conjugate comprising bisphosphonate (BP) conjugated to an antibody for the treatment or prevention of bone tumors in a subject with cancer.

In some aspects, the subject has bone cancer or bone metastasis. In particular aspects, the bone cancer is Ewing sarcoma, osteosarcoma, or chondrosarcoma. In certain aspects, the bone metastasis is from breast cancer, myeloma, renal cancer, lung cancer, prostate cancer, thyroid cancer, or bladder cancer. In specific aspects, the bone metastasis is breast cancer bone metastasis. In some aspects, the breast cancer is triple-negative breast cancer. In certain aspects, the breast cancer is HER2-negative breast cancer. In other aspects, the breast cancer is HER2-positive breast cancer.

In certain aspects, the BP is negatively-charged. In some aspects, the BP is alendronate, zoledronate, pamidronate, risedronate, medronic acid, aminomethylene bisphonic acid, clodronate, etidronate, tiludronate, ibandronate pomidronate, neridonate, olpadronate, or oxidronate. In particular aspects, the BP is alendronate (ALN).

In some aspects, the antibody is a monoclonal antibody, bispecific antibody, Fab′, a F(ab′)2, a F(ab′)3, a monovalent scFv, a bivalent scFv, a single domain antibody, or nanobody. In certain aspects, the antibody is an immune checkpoint inhibitor. In particular aspects, the antibody is an anti-HER2 antibody, anti-CD99 antibody, anti-IGF-IR antibody, anti-PD-L1, anti-PD-1, anti-CTLA4-antibody, anti-Siglec-15 antibody, anti-RANKL antibody, or anti-TGFβ antibody. In specific aspects, the antibody is an anti-HER2 antibody, such as trastuzumab (Herceptin), pertuzumab (Perjeta), or atezolizumab. In particular aspects, the antibody is trastuzumab. In some aspects, bone-targeting conjugate comprises alendronate conjugated to trastuzumab. In certain aspects, the antibody is not an anti-M-CSF antibody.

In certain aspects, the BP is not conjugated to N-glycan on the Fc region of the antibody. In some aspects, the BP is site-specifically conjugated to the antibody using pClick conjugation, NHS-ester chemistry, or cysteine chemistry. In particular aspects, the BP is site-specifically conjugated to the antibody using pClick conjugation. In some aspects, the BP is conjugated to the CH2-CH3 junction of the antibody. In some aspects, the BP is conjugated to the antibody using 4-fluorophenyl carbamate lysine (FPheK). In particular aspects, FPheK is attached to a fragment of the B domain of protein A (FB protein) from Staphylococcus aureus. In specific aspects, pClick conjugation comprises conjugation of an antibody with an azide functional moiety with BP functionalized with bicyclo[6.1.0]nonyne (BCN).

In certain aspects, the bone-targeting conjugate results in increased concentration of therapeutic antibody at the bone tumor niche, inhibits cancer development in the bone, and/or limits secondary metastases to other organs. In some aspects, the bone-targeting conjugate results in decreased micrometastasis-induced osteolyic lesions.

In additional aspects, the use further comprises an additional anti-cancer therapy. In some aspects, the additional anti-cancer therapy comprises surgery, chemotherapy, radiation therapy, hormonal therapy, immunotherapy or cytokine therapy. In particular aspects, the additional anti-cancer therapy comprises immunotherapy or chemotherapy.

It is contemplated that any method or composition described herein can be implemented with respect to any other method or composition described herein. For example, a compound synthesized by one method may be used in the preparation of a final compound according to a different method.

Other objects, features and advantages of the present disclosure will become apparent from the following detailed description. It should be understood, however, that the detailed description and the specific examples, while indicating specific embodiments of the disclosure, are given by way of illustration only, since various changes and modifications within the spirit and scope of the disclosure will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-1L: (A) Therapeutic antibodies can be site-specifically delivered to bone by pClick conjugation of bisphosphonate molecules that bind to the bone hydroxyapatite matrix. (B) SDS-PAGE analysis of Tras, Tras-ALN, and their near-infrared (NIR) fluorophore conjugates under reducing and non-reducing conditions, visualized by coomassie blue staining (left) and a fluorescence scanner (right) (C) Mass spectrometry analysis of Tras and Tras-ALN. (D) Flow cytometric profiles of Tras and Tras-ALN binding to BT474 (HER2+++), SK-BR-3 (HER2+++), MDA-MB-361 (HER2++), and MDA-MB-468 (HER2−) cells. (E-G) In vitro cytotoxicity of Tras and Tras-ALN against BT474, MDA-MB-361, and MDA-MB-468 cells. (H) Differential bone targeting ability of unmodified Tras and Tras-ALN conjugate. Nondecalcified bone sections from C57/BL6 mice were incubated with 50 μg/mL Tras or Tras-ALN overnight, followed by staining with fluorescein isothiocyanate (FITC)-labeled anti-human IgG and 4 μg/mL xylenol orange (XO, known to label bone), Scale bars, 200 μm. (I-J) Binding kinetics of Tras and Tras-ALN to hydroxyapatite (HA) and native bone. (K) Ex vivo fluorescence images of lower limbs of athymic nude mice bearing MDA-MB-361 tumors 24 h, 96 h, or 168 h after the retro-orbital injection of Cy7.5-labeled Tras and Tras-ALN. Tumor cells were inoculated into the right limbs of nude mice via IIA injection. (L) Nondecalcified bone sections from the biodistribution study were stained with FITC-labeled anti-human IgG (green), RFP (red) and DAPI (blue), Scale bars, 100 μm.

FIGS. 2A-2N: Tras-ALN inhibits breast cancer micrometastases in the bone. (A) MDA-MB-361 cells were IIA injected into the right hind limb of nude mice, followed by treatment with PBS, ALN (10 μg/kg retro-orbital venous sinus in PBS twice a week), Tras (1 mg/kg retro-orbital venous sinus in sterile PBS twice a week), and Tras-ALN conjugate (same as Tras). Tumor burden was monitored by weekly bioluminescence imaging. (B) Fold-change in mean luminescent intensity of MDA-MB-361 tumors in mice treated as described in (A), two-way ANOVA comparing Tras to Tras-ALN. (C) Fold-change in Individual luminescent intensity of HER2-positive MDA-MB-361 tumors in mice treated as described in (A). (D) Kaplan-Meier plot of the time-to-euthanasia of mice treated as described in (A). For each individual mouse, the BLI signal in the whole body reached 10⁷ photons sec⁻¹ was considered as the endpoint. (E) Body weight change of tumor-bearing mice over time. (F) MicroCT scanning in the supine position for groups treated with PBS, ALN, Tras, or Tras-ALN 82 days after tumor implantation. (G) Quantitative analysis of bone volume density (BV/TV). (H) Quantitative analysis of trabecular thickness (Tb.Th). (I) Quantitative analysis of trabecular bone mineral density (BMD). (J) Representative longitudinal, midsagittal hematoxylin and eosin (H&E)-stained sections of tibia/femur from each group. T: tumor; B: bone; BM: bone marrow. (K) Representative images of HER2 and TRAP staining of bone sections from each group. (L) Osteoclast number per image calculated at the tumor-bone interface in each group (pink cells in (K) were considered as osteoclast positive cells). (M) Serum TRAcP 5b levels of mice treated as described in (A). (N) Serum calcium levels of mice treated as described in (A). ****P<0.0001, ***P<0.001, **P<0.01, *P<0.05, and n.s.=P>0.05.

FIGS. 3A-3C: (A) Secondary metastases observed in various organs in mice treated with Tras (top) or Tras-ALN (bottom). (B) Pie charts show the frequencies of metastasis observed in various organs in mice treated with Tras (1 mg/kg retro-orbitally in sterile PBS twice a week), and Tras-ALN conjugate (same as Tras). (C) Quantification of bioluminescence signal intensity in different organs, including other bones, as measurement of metastases resulted from Tras and Tras-ALN-treated mice. p values are based on one-way ANOVA test. *P<0.05 and n.s.=P>0.05.

FIGS. 4A-4E: In vivo comparison of Tras and Tras-ALN in HER2-negative model. (A) Tumor burden was monitored by weekly bioluminescence imaging, and (B) quantified by the radiance detected in the region of interest. (C) Fold-change in Individual luminescent intensity of HER2-negative MCF-7 tumors in mice treated as described in (A). (D) Kaplan-Meier plot of the time-to-sacrifice of mice treated as described in (A). For each individual mouse, the BLI signal in the whole body reached 10⁷ photons sec⁻¹ was considered as the endpoint. (E) Body weight change of tumor-bearing mice over time. ****P<0.0001, *P<0.05, and n.s.=P>0.05.

FIG. 5: ESI-MS spectra of BCN-ALN.

FIG. 6: ESI-MS spectra of ssFB-FPheK.

FIG. 7: ESI-MS spectra of Tras-azide.

FIGS. 8A-8H: Tras binding to BT474 cells. BT474 cells were incubated with increasing concentrations of Tras and process as described in Methods and fluorescence was measured on the flow cytometer. The KD was determined as follows: 1/F=1/Fmax+(KD/Fmax)(1/[Ab].

FIGS. 9A-9H: Tras-ALN binding to BT474 cells. BT474 cells were incubated with increasing concentrations of Tras-ALN and process as described in Methods, and fluorescence was measured on the flow cytometer. The KD was determined as follows: 1/F=1/Fmax+(KD/Fmax)(1/[Ab].

FIGS. 10A-10H: Tras binding to SK-BR-3 cells. SK-BR-3 cells were incubated with increasing concentrations of Tras and process as described in Methods and fluorescence was measured on the flow cytometer. The KD was determined as follows: 1/F=1/Fmax+(KD/Fmax)(1/[Ab].

FIGS. 11A-11H: Tras-ALN binding to SK-BR-3 cells. SK-BR-3 cells were incubated with increasing concentrations of Tras and process as described in Methods and fluorescence was measured on the flow cytometer. The KD was determined as follows: 1/F=1/Fmax+(KD/Fmax)(1/[Ab].

FIG. 12: Binding of Tra-ALN in BT-474, SK-BR-3, and MDA-MB-468 cells visualized by confocal microscopy. Cells were incubated with 30 nM Tras-ALN in media for 30 min at 37° C. and stained with DilC18 (red fluorescence) and Hoechst nuclear stain (blue fluorescence).

FIGS. 13A-13D: Ex vivo fluorescence images of main organs. Heart, liver, spleen, lung, kidney, brain of athymic nude mice bearing MDA-MB-361 tumors 96 h after the retro-orbital injection Cy7.5-labeled (A) Tras and (B) Tras-ALN. (C) Heart, liver, spleen, lung, kidney and bones of tumor bearing mice MDA-MB-361 tumors 72 h after the retro-orbital injection Cy7.5-labeled Tras and Tras-ALN. (D) Pharmacokinetic profiles of Tras and Tras-ALN. Tumor bearing athymic nude mice (3 months after surgery) were injected retro-orbitally with 1 mg/kg Tras and Tras-ALN in PBS. Antibody concentrations in the serum were determined by ELISA (Data represent the mean±SD for three independent repeats).

FIG. 14: Ex vivo fluorescence images analysis for the bone biodistribution of Tras and Tras-ALN. 24 h, 96 h or 168 h after after the retro-orbital injection of Cy7.5-labeled Tras and Tras-ALN. The bone was collected and analysis. The quantity data was summarized from FIG. 1K. The signal of free tumor from Tras treated mice was considered as blank. The Relative signal was calculated as follows: The signal from hind limbs—The signal from free tumor hind limbs (from Tras treated group).

FIG. 15: Tras-ALN inhibits breast cancer micrometastases in the bone. MDA-MB-361 cells were IIA injected into the right hind limb of nude mice, followed by treatment with PBS, ALN (10 μg/kg retro-orbital venous sinus in PBS twice a week), Tras (1 mg/kg retro-orbital venous sinus in sterile PBS twice a week), and Tras-ALN conjugate (same as Tras). Tumor burden was monitored twice a week by bioluminescence imaging (Day 6, 20, 33, 48 and 68 imaging data were selected to show in FIG. 2A).

FIGS. 16A-16B: Whole body BLI quantification. (A) The BLI from each treatment group quantified by the radiance detected in the whole body. (B) Individual whole body luminescent intensity of different treated group as described in FIG. 2A. ****P<0.0001.

FIGS. 17A-17D: BLI signals in the hind limbs were quantified in Tras and Tras-ALN treated group and are shown. (A) Fold-change in mean luminescent intensity of hind limbs in mice treated with Tras and Tras-ALN (as described in FIG. 2A), two-way ANOVA comparing Tras to Tras-ALN. (B) Fold-change in Individual luminescent intensity of hind limbs in Tras and Tras-ALN treated group. (C) The mean BLI of hind limbs from Tras and Tras-ALN treatment group quantified. (D) Individual luminescent intensity of Tras and Tras-ALN treated group. ****P<0.0001.

FIG. 18: MicroCT-based 3D renderings of bones. Cortical bone, images show extensive cortical bone destruction. Trabecular bone, images show trabecular destruction. Lower panel (growth plate), images plate show bone loss at growth plate.

FIG. 19: TRAP staining of bone sections from each group.

FIGS. 20A-20E: The in vivo quantification of secondary metastases. (A) BLI signal in the whole body and hind limbs of mice in various treatment groups were quantified and are shown. The secondary metastases was determined as follows: BLI signals in whole body and the hind limbs (shown by red circles) were quantified. Each time point, animals were imaged twice a week using IVIS Lumina II (Advanced Molecular Vision), following the recommended procedures and manufacturer's settings. For the groups which signal suggested “Saturated Luminescent Image”, it will be scanned for shorter time (which were indicated under the imaging). The secondary metastases were calculated as follows: BLI signal intensity in whole body—BLI signal intensity in hind limbs. (B) Fold-change in mean luminescent intensity of secondary metastases in mice treated as described in (FIG. 2A), two-way ANOVA comparing Tras to Tras-ALN. (C) Fold-change in Individual luminescent intensity of secondary metastases in Tras and Tras-ALN treated group. (D) The mean BLI of secondary metastases from Tras and Tras-ALN treatment group quantified. (E) Individual luminescent intensity of Tras and Tras-ALN treated group. ****P<0.0001.

FIG. 21: Secondary metastases observed in various organs in mice treated with Tras (top) or Tras-ALN (bottom).

FIGS. 22A-22C: In vivo comparison of Tras and Tras-ALN in HER2-negative model. MCF-7 cells were IIA injected into the right hind limb of nude mice, followed by treatment with Tras (1 mg/kg retro-orbital venous sinus in sterile PBS twice a week), and Tras-ALN conjugate (same as Tras). (A) Tumor burden was monitored twice a week by bioluminescence imaging (Day 4, 12, 19, 29 and 42 imaging data were selected to show in FIG. 4A. (B) The BLI from each treatment group quantified by the radiance detected in the whole body. (C) Individual whole body luminescent intensity of Tras and Tras-ALN treated group. ****P<0.0001.

FIGS. 23A-23D: BLI signals in the hind limbs in MCF-7 model were quantified in Tras and Tras-ALN treated groups and are shown. (A) Fold-change in mean luminescent intensity of hind limbs in mice treated with Tras and Tras-ALN (as described in FIG. 4A), two-way ANOVA comparing Tras to Tras-ALN. (B) Fold-change in Individual luminescent intensity of hind limbs in Tras and Tras-ALN treated groups. (C) The mean BLI of hind limbs from Tras and Tras-ALN treatment groups quantified. (D) Individual luminescent intensity of Tras and Tras-ALN treated groups. ****P<0.0001.

FIGS. 24A-24B: Effects of Tras-ALN on MCF-7 HER2-negative model: serum TRACP 5b and calcium levels analysis. (A) Serum TRACP 5b concentration in Tras and Tras-ALN at the end of experiment (*P<0.05). (B) Serum calcium concentration in Tras and Tras-ALN group at the end of experiment (*P<0.05).

FIGS. 25A-25E: The in vivo quantification of secondary metastases. (A) BLI signal in the whole body and hind limbs of mice in various treatment groups were quantified and are shown. The secondary metastases were determined as follows: BLI signals in whole body and the hind limbs (shown by red circles) were quantified. Each time point, animals were imaged twice a week using IVIS Lumina II (Advanced Molecular Vision), following the recommended procedures and manufacturer's settings. For the groups which signal suggested “Saturated Luminescent Image”, it will be scanned for shorter time (which were indicated under the imaging). The secondary metastases were calculated as follows: BLI signal intensity in whole body—BLI signal intensity in hind limbs. (B) Fold-change in mean luminescent intensity of secondary metastases in mice treated as described in FIG. 22, two-way ANOVA comparing Tras to Tras-ALN. (C) Fold-change in Individual luminescent intensity of secondary metastases in Tras and Tras-ALN treated groups. (D) The mean BLI of secondary metastases from each treatment group quantified. (E) Individual luminescent intensity of Tras and Tras-ALN treated groups. ****P<0.0001.

FIGS. 26A-26B: Tras-ALN effects on multi-organs metastases in MCF-7 cell lines. (A) Metastases observed in various organs in mice treated with Tras or Tras-ALN. (B) Pie charts show the frequencies of metastasis observed in various organs in Tras and Tras-ALN treated groups.

FIGS. 27A-27C: Therapeutic effect of Tras-ALN on the mice with both bone metastases and primary tumor. (A) Luciferase labeled MDA-MB-361 cells were injected into the right hind limb using IIA injection. Non-luciferase labeled MDA-MB-361 cells were inoculated at mammary fat pad on the same mice. Then the mice were treated with PBS (n=5), Tras (1 mg/kg retro-orbital venous sinus in sterile PBS, n=7), and Tras-ALN (same as Tras, n=7). Tumor burden at hind limb was monitored by bioluminescence imaging (BLI). Tumor burden at mammary fat pad was measured using vernier caliper. (B) Hind limb tumor fold-change in mean luminescent intensity in mice treated as described in (A), two-way ANOVA comparing hind limb tumor of Tras and Tras-ALN groups. (C) Mammary fat pad tumor fold-change in mean luminescent intensity in mice treated as described in (A), two-way ANOVA comparing mammary fat pad tumor of Tras and Ttras-ALN groups. *P<0.05, and n.s. represents P>0.05.

### DESCRIPTION OF ILLUSTRATIVE EMBODIMENTS

Over the past 20 years, antibody-based therapies have proved to be of great value in cancer treatment. Despite the clinical success of these biopharmaceuticals, reaching targets in the bone microenvironment has proved to be difficult perhaps due to the relatively low vascularization of bone tissue and the presence of physical barriers that impair drug penetration. Attempts to ensure effective concentrations of a therapeutic drug in bone unavoidably lead to high concentrations in other tissues as well, often resulting in adverse systemic effects or side effects that may limit or exclude the use of the drug.^{24,25} In this case, the potential benefit of passive targeting is lost.

Accordingly, in certain embodiments, the present disclosure provides an innovative bone targeting (BonTarg) technology that enables the specific delivery of therapeutic polypeptides, such as antibodies, to the bone via conjugation of bone-targeting moieties. The resulting bone-targeting antibodies can specifically target the bone metastatic niche to eliminate bone micrometastases and also prevent seeding of multi-organ metastases from bone lesions. Taking advantage of the high mineral concentration unique to the bone hydroxyapatite matrix, bisphosphonate (BP) conjugation has been used for selective delivery of small molecule drugs, imaging probes, nuclear medicines, and nanoparticles to the bone as a means of treating of osteoporosis, primary and metastatic bone neoplasms, and other bone disorders.^{24,26-30} Negatively-charged BP has a high affinity for mineralized, positively charged bone matrix, such as hydroxyapatite (HA), which is the main component of hard bone, resulting in preferential binding to the bone. Thus, in certain aspects, the present methods comprise the use of pClick conjugation technology to site-specifically couple a BP drug, such as Alendronate (ALN), to an antibody, such as the HER2-targeting monoclonal antibody trastuzumab (Tras).³¹ The present studies showed that in two xenograft models based on intra-iliac artery (IIA) injection, the resulting trastuzumab-Alendronate conjugate (Tras-ALN) significantly enhanced the concentration of therapeutic antibody in the bone metastatic niche, inhibited cancer development in the bone, and limited secondary metastases to other organs. This type of specific delivery of therapeutic antibodies to the bone has the potential to enhance both the breadth and potency of antibody therapy for bone-related diseases.

### I. Definitions

As used herein, “essentially free,” in terms of a specified component, is used herein to mean that none of the specified component has been purposefully formulated into a composition and/or is present only as a contaminant or in trace amounts. The total amount of the specified component resulting from any unintended contamination of a composition is therefore well below 0.05%, preferably below 0.01%. Most preferred is a composition in which no amount of the specified component can be detected with standard analytical methods.

As used herein the specification, “a” or “an” may mean one or more. As used herein in the claim(s), when used in conjunction with the word “comprising,” the words “a” or “an” may mean one or more than one.

The use of the term “or“in the claims is used to mean” and/or” unless explicitly indicated to refer to alternatives only or the alternatives are mutually exclusive, although the disclosure supports a definition that refers to only alternatives and “and/or.” As used herein “another” may mean at least a second or more.

The term “about” means, in general, within a standard deviation of the stated value as determined using a standard analytical technique for measuring the stated value. The terms can also be used by referring to plus or minus 5% of the stated value.

The phrase “effective amount” or “therapeutically effective” means a dosage of a drug or agent sufficient to produce a desired result. The desired result can be subjective or objective improvement in the recipient of the dosage, increased lung growth, increased lung repair, reduced tissue edema, increased DNA repair, decreased apoptosis, a decrease in tumor size, a decrease in the rate of growth of cancer cells, a decrease in metastasis, or any combination of the above.

As used herein, the term “antibody” refers to an immunoglobulin, derivatives thereof which maintain specific binding ability, and proteins having a binding domain which is homologous or largely homologous to an immunoglobulin binding domain. These proteins may be derived from natural sources, or partly or wholly synthetically produced. An antibody may be monoclonal or polyclonal. The antibody may be a member of any immunoglobulin class, including any of the human classes: IgG, IgM, IgA, IgD, and IgE. The antibody may be a bi-specific antibody. In exemplary embodiments, antibodies used with the methods and compositions described herein are derivatives of the IgG class. The term antibody also refers to antigen-binding antibody fragments. Examples of such antibody fragments include, but are not limited to, Fab, Faby, F(abÿ)2, scFv, Fv, dsFv diabody, and Fd fragments. Antibody fragment may be produced by any means. For instance, the antibody fragment may be enzymatically or chemically produced by fragmentation of an intact antibody, it may be recombinantly produced from a gene encoding the partial antibody sequence, or it may be wholly or partially synthetically produced. The antibody fragment may optionally be a single chain antibody fragment. Alternatively, the fragment may comprise multiple chains which are linked together, for instance, by disulfide linkages. The fragment may also optionally be a multimolecular complex. A functional antibody fragment will typically comprise at least about 10 amino acids and more typically will comprise at least about 200 amino acids.

“Subject” and “patient” refer to either a human or non-human, such as primates, mammals, and vertebrates. In particular embodiments, the subject is a human.

As used herein, the terms “treat,” “treatment,” “treating,” or “amelioration” when used in reference to a disease, disorder or medical condition, refer to therapeutic treatments for a condition, wherein the object is to reverse, alleviate, ameliorate, inhibit, slow down or stop the progression or severity of a symptom or condition. The term “treating” includes reducing or alleviating at least one adverse effect or symptom of a condition. Treatment is generally “effective” if one or more symptoms or clinical markers are reduced. Alternatively, treatment is “effective” if the progression of a condition is reduced or halted. That is, “treatment” includes not just the improvement of symptoms or markers, but also a cessation or at least slowing of progress or worsening of symptoms that would be expected in the absence of treatment. Beneficial or desired clinical results include, but are not limited to, alleviation of one or more symptom(s), diminishment of extent of the deficit, stabilized (i.e., not worsening) state of a tumor or malignancy, delay or slowing of tumor growth and/or metastasis, and an increased lifespan as compared to that expected in the absence of treatment.

### II. Bone-Targeting Antibody Conjugate

The present disclosure relates to conjugation of a bone-targeting moiety, such as bisphosphonate, to an antibody. In some aspects, the bone-targeting agent may be conjugated to the antibody at a site far from the antigen binding site and Fc receptor binding site, such as the CH2-CH3 junction.

Bisphosphonates are synthetic compounds containing two phosphonate groups bound to a central (geminal) carbon (the P-C-P backbone) that are used to prevent bone resorption in a number of metabolic and tumor-induced bone diseases including multiple myeloma. Bisphosphonate treatment is associated with an increase in patient survival, indicating that these compounds have a direct effect on the tumor cells. Bisphosphonates may contain two additional chains bound to the central geminal carbon. The presence of these two side chains allows numerous substitutions to the bisphosphonate backbone and therefore the development of a variety of analogs with different pharmacological properties. Exemplary bisphosphonates include but are not limited to alendronate, zoledronate, pamidronate, risedronate, medronic acid, aminomethylene bisphonic acid, clodronate, etidronate, tilundronate, or ibandronate.

In certain aspects, the bone-targeting agent may be conjugated to the antibody by site-specific conjugation methods. In one method, the antibody is conjugated by cysteine chemistry comprising engineered cysteine substitutions at positions on the light and heavy chains that provide reactive thiol groups and do not perturb immunoglobulin folding and assembly, or alter antigen binding (Junutula et a., 2008; incorporated herein by reference in its entirety). In another method, the conjugation method comprises site-specific introduction of aldehyde groups into recombinant proteins using the 6-amino-acid consensus sequence recognized by the formylglycine-generating enzyme (Carrico et al., 2007; incorporated herein by reference in its entirety). This genetically encoded ‘aldehyde tag’ is no larger than a His6 tag and can be exploited for numerous protein labeling applications. In some aspects, the site-specific conjugation method comprises remodeled Fc N-glycans of antibodies using mutant glycosyltransferases, such as mutant beta1,4-galactosyltransferase (Boeggeman et al., 2009; incorporated herein by reference in its entirety) or transglutaminase-mediated site-specific conjugation. In specific aspects, the site-specific conjugation comprises use of disulfide bridges (Zhang et al., 2016; incorporated herein by reference in its entirety). In certain aspects, the site-specific conjugation method comprises incorporation of non-canonical amino acids (Leisle et al., 2015; incorporated herein by reference in its entirety). For example, the bone-targeting agent may be conjugated to the antibody using pClick technology comprising proximity-induced site-specific conjugation using an affinity compound (WO2019/217900; incorporated herein by reference in its entirety). pClick is a site-specific technology that doesn't require the antibody engineering and any chemical or enzymatic treatments. The pClick method can enable site-specific covalent bond formation between the bone-targeting moiety and the antibody. In particular aspects, the bone-targeting antibody conjugate does not comprise a polymeric backbone. In specific aspects, the bone-targeting moiety is not conjugated to the N-glycan of the Fc domain of the antibody.

Specifically, the present methods may comprise proximity-induced reactivity between an ncAA and a nearby antibody residue, such as a lysine or cysteine. pClick can enable covalent bond formation between the bone-targeting moiety and a defined residue, such as lysine, of the antibody without performing antibody engineering.

The present antibody conjugated may be further conjugated to an imaging or diagnostic agent.

A “therapeutic agent” as used herein refers to any agent that can be administered to a subject for the purpose of obtaining a therapeutic benefit of a disease or health-related condition. For example, antibodies conjugated to a therapeutic agent may be administered to a subject for the purpose of reducing the size of a tumor, reducing or inhibiting local invasiveness of a tumor, or reducing the risk of development of metastases.

A “diagnostic agent” or “imaging agent” (referred to interchangeably) as used herein refers to any agent that can be administered to a subject for the purpose of diagnosing a disease or health-related condition in a subject. Diagnosis may involve determining whether a disease is present, whether a disease has progressed, or any change in disease state.

The therapeutic or diagnostic agent may be a small molecule, a peptide, a protein, a polypeptide, an antibody, an antibody fragment, a DNA, or an RNA.

### A. Formulation and Administration

The present disclosure provides pharmaceutical compositions comprising antibodies conjugated to a bone-targeting agent, such as bisphosphonate. Such compositions comprise a prophylactically or therapeutically effective amount of an antibody or a fragment thereof, or a peptide immunogen, and a pharmaceutically acceptable carrier. In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a particular carrier when the pharmaceutical composition is administered intravenously. Saline solutions and aqueous dextrose and glycerol solutions can also be employed as liquid carriers, particularly for injectable solutions. Other suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like.

The composition, if desired, can also contain minor amounts of wetting or emulsifying agents, or pH buffering agents. These compositions can take the form of solutions, suspensions, emulsion, tablets, pills, capsules, powders, sustained-release formulations and the like. Oral formulations can include standard carriers such as pharmaceutical grades of mannitol, lactose, starch, magnesium stearate, sodium saccharine, cellulose, magnesium carbonate, etc. Examples of suitable pharmaceutical agents are described in “Remington's Pharmaceutical Sciences.” Such compositions will contain a prophylactically or therapeutically effective amount of the antibody or fragment thereof, preferably in purified form, together with a suitable amount of carrier so as to provide the form for proper administration to the patient. The formulation should suit the mode of administration, which can be oral, intravenous, intraarterial, intrabuccal, intranasal, nebulized, bronchial inhalation, or delivered by mechanical ventilation.

Active vaccines are also envisioned where antibodies like those disclosed are produced in vivo in a subject at risk of Poxvirus infection. Such vaccines can be formulated for parenteral administration, e.g., formulated for injection via the intradermal, intravenous, intramuscular, subcutaneous, or even intraperitoneal routes. Administration by intradermal and intramuscular routes are contemplated. The vaccine could alternatively be administered by a topical route directly to the mucosa, for example by nasal drops, inhalation, or by nebulizer. Pharmaceutically acceptable salts, include the acid salts and those which are formed with inorganic acids such as, for example, hydrochloric or phosphoric acids, or such organic acids as acetic, oxalic, tartaric, mandelic, and the like. Salts formed with the free carboxyl groups may also be derived from inorganic bases such as, for example, sodium, potassium, ammonium, calcium, or ferric hydroxides, and such organic bases as isopropylamine, trimethylamine, 2-ethylamino ethanol, histidine, procaine, and the like.

Passive transfer of antibodies, known as artificially acquired passive immunity, generally will involve the use of intravenous or intramuscular injections. The forms of antibody can be human or animal blood plasma or serum, as pooled human immunoglobulin for intravenous (IVIG) or intramuscular (IG) use, as high-titer human IVIG or IG from immunized or from donors recovering from disease, and as monoclonal antibodies (MAb). Such immunity generally lasts for only a short period of time, and there is also a potential risk for hypersensitivity reactions, and serum sickness, especially from gamma globulin of non-human origin. However, passive immunity provides immediate protection. The antibodies will be formulated in a carrier suitable for injection, i.e., sterile and syringeable.

Generally, the ingredients of compositions of the disclosure are supplied either separately or mixed together in unit dosage form, for example, as a dry lyophilized powder or water-free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The compositions of the disclosure can be formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

### B. Hyperproliferative Diseases

While hyperproliferative diseases can be associated with any disease which causes a cell to begin to reproduce uncontrollably, the prototypical example is cancer. One of the key elements of cancer is that the cell's normal apoptotic cycle is interrupted and thus agents that interrupt the growth of the cells are important as therapeutic agents for treating these diseases. In this disclosure, a bone-targeting antibody conjugate may be used to treat a variety of types of cancers, such as bone cancers and cancers that metastasize to the bone.

Cancer cells that may be treated with the compounds of the present disclosure include but are not limited to cells from the bladder, blood, bone, bone marrow, brain, breast, colon, esophagus, gastrointestine, gum, head, kidney, liver, lung, nasopharynx, neck, ovary, prostate, skin, stomach, pancreas, testis, tongue, cervix, or uterus. In addition, the cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; androblastoma, malignant; sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malig melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; hodgkin's disease; hodgkin's; paragranuloma; malignant lymphoma, small lymphocytic; malignant lymphoma, large cell, diffuse; malignant lymphoma, follicular; mycosis fungoides; other specified non-hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia. In certain aspects, the tumor may comprise an osteosarcoma, angiosarcoma, rhabdosarcoma, leiomyosarcoma, Ewing sarcoma, glioblastoma, neuroblastoma, or leukemia.

### C. Methods of Treatment

In particular, compositions that may be used in treating cancer in a subject (e.g., a human subject) are disclosed herein. The compositions described above are preferably administered to a mammal (e.g., rodent, human, non-human primates, canine, bovine, ovine, equine, feline, etc.) in an effective amount, that is, an amount capable of producing a desirable result in a treated subject (e.g., causing apoptosis of cancerous cells or killing bacterial cells). Toxicity and therapeutic efficacy of the compositions utilized in methods of the disclosure can be determined by standard pharmaceutical procedures. As is well known in the medical and veterinary arts, dosage for any one animal depends on many factors, including the subject's size, body surface area, body weight, age, the particular composition to be administered, time and route of administration, general health, the clinical symptoms of the infection or cancer and other drugs being administered concurrently. A composition as described herein is typically administered at a dosage that inhibits the growth or proliferation of a bacterial cell, inhibits the growth of a biofilm, or induces death of cancerous cells (e.g., induces apoptosis of a cancer cell), as assayed by identifying a reduction in hematological parameters (Complete blood count (CBC)), or cancer cell growth or proliferation.

The therapeutic methods of the disclosure (which include prophylactic treatment) in general include administration of a therapeutically effective amount of the compositions described herein to a subject in need thereof, including a mammal, particularly a human. Such treatment will be suitably administered to subjects, particularly humans, suffering from, having, susceptible to, or at risk for a disease, disorder, or symptom thereof. Determination of those subjects “at risk” can be made by any objective or subjective determination by a diagnostic test or opinion of a subject or health care provider (e.g., genetic test, enzyme or protein marker, marker (as defined herein), family history, and the like).

In one embodiment, the disclosure provides a method of monitoring treatment progress. The method includes the step of determining a level of changes in hematological parameters and/or cancer stem cell (CSC) analysis with cell surface proteins as diagnostic markers (which can include, for example, but are not limited to CD34, CD38, CD90, and CD117) or diagnostic measurement (e.g., screen, assay) in a subject suffering from or susceptible to a disorder or symptoms thereof associated with cancer (e.g., leukemia) in which the subject has been administered a therapeutic amount of a composition as described herein. The level of marker determined in the method can be compared to known levels of marker either in healthy normal controls or in other afflicted patients to establish the subject's disease status. In preferred embodiments, a second level of marker in the subject is determined at a time point later than the determination of the first level, and the two levels are compared to monitor the course of disease or the efficacy of the therapy. In certain preferred embodiments, a pre-treatment level of marker in the subject is determined prior to beginning treatment according to the methods described herein; this pre-treatment level of marker can then be compared to the level of marker in the subject after the treatment commences, to determine the efficacy of the treatment.

### D. Additional Therapy

In certain embodiments, the compositions and methods of the present embodiments involve a bone-targeting antibody conjugate, in combination with a second or additional therapy. Such therapy can be applied in the treatment of any disease with bone tumors. For example, the disease may be a bone cancer or bone metastasis.

In certain embodiments, the compositions and methods of the present embodiments involve a bone-targeting antibody conjugate in combination with at least one additional therapy. The additional therapy may be radiation therapy, surgery (e.g., lumpectomy and a mastectomy), chemotherapy, gene therapy, DNA therapy, viral therapy, RNA therapy, immunotherapy, bone marrow transplantation, nanotherapy, monoclonal antibody therapy, or a combination of the foregoing. The additional therapy may be in the form of adjuvant or neoadjuvant therapy.

The methods and compositions, including combination therapies, enhance the therapeutic or protective effect, and/or increase the therapeutic effect of another anti-cancer or anti-hyperproliferative therapy. Therapeutic and prophylactic methods and compositions can be provided in a combined amount effective to achieve the desired effect, such as the killing of a cancer cell and/or the inhibition of cellular hyperproliferation. This process may involve contacting the cells with both an antibody or antibody fragment and a second therapy. A tissue, tumor, or cell can be contacted with one or more compositions or pharmacological formulation(s) comprising one or more of the agents (i.e., antibody or antibody fragment or an anti-cancer agent), or by contacting the tissue, tumor, and/or cell with two or more distinct compositions or formulations, wherein one composition provides 1) an antibody or antibody fragment, 2) an anti-cancer agent, or 3) both an antibody or antibody fragment and an anti-cancer agent. Also, it is contemplated that such a combination therapy can be used in conjunction with chemotherapy, radiotherapy, surgical therapy, or immunotherapy.

The terms “contacted” and “exposed,” when applied to a cell, are used herein to describe the process by which a therapeutic construct and a chemotherapeutic or radiotherapeutic agent are delivered to a target cell or are placed in direct juxtaposition with the target cell. To achieve cell killing, for example, both agents are delivered to a cell in a combined amount effective to kill the cell or prevent it from dividing.

An inhibitory antibody may be administered before, during, after, or in various combinations relative to an anti-cancer treatment. The administrations may be in intervals ranging from concurrently to minutes to days to weeks. In embodiments where the antibody or antibody fragment is provided to a patient separately from an anti-cancer agent, one would generally ensure that a significant period of time did not expire between the time of each delivery, such that the two compounds would still be able to exert an advantageously combined effect on the patient. In such instances, it is contemplated that one may provide a patient with the antibody therapy and the anti-cancer therapy within about 12 to 24 or 72 h of each other and, more particularly, within about 6-12 h of each other. In some situations, it may be desirable to extend the time period for treatment significantly where several days (2, 3, 4, 5, 6, or 7) to several weeks (1, 2, 3, 4, 5, 6, 7, or 8) lapse between respective administrations.

In certain embodiments, a course of treatment will last 1-90 days or more (this such range includes intervening days). It is contemplated that one agent may be given on any day of day 1 to day 90 (this such range includes intervening days) or any combination thereof, and another agent is given on any day of day 1 to day 90 (this such range includes intervening days) or any combination thereof. Within a single day (24-hour period), the patient may be given one or multiple administrations of the agent(s). Moreover, after a course of treatment, it is contemplated that there is a period of time at which no anti-cancer treatment is administered. This time period may last 1-7 days, and/or 1-5 weeks, and/or 1-12 months or more (this such range includes intervening days), depending on the condition of the patient, such as their prognosis, strength, health, etc. It is expected that the treatment cycles would be repeated as necessary.

In some embodiments, the additional therapy is the administration of small molecule enzymatic inhibitor or anti-metastatic agent. In some embodiments, the additional therapy is the administration of side-effect limiting agents (e.g., agents intended to lessen the occurrence and/or severity of side effects of treatment, such as anti-nausea agents, etc.). In some embodiments, the additional therapy is radiation therapy. In some embodiments, the additional therapy is surgery. In some embodiments, the additional therapy is a combination of radiation therapy and surgery. In some embodiments, the additional therapy is gamma irradiation. In some embodiments, the additional therapy is therapy targeting PBK/AKT/mTOR pathway, HSP90 inhibitor, tubulin inhibitor, apoptosis inhibitor, and/or chemopreventative agent. The additional therapy may be one or more of the chemotherapeutic agents known in the art.

Various combinations may be employed. For the example below a bone-targeting antibody conjugate, is “A” and an anti-cancer therapy is “B”:
A/B/A B/A/B B/B/A A/A/B A/B/B B/A/A A/B/B/B B/A/B/B
B/B/B/A B/B/A/B A/A/B/B A/B/A/B A/B/B/A B/B/A/A
B/A/B/A B/A/A/B A/A/A/B B/A/A/A A/B/A/A A/A/B/A

Administration of any compound or therapy of the present embodiments to a patient will follow general protocols for the administration of such compounds, taking into account the toxicity, if any, of the agents. Therefore, in some embodiments there is a step of monitoring toxicity that is attributable to combination therapy.

### 1. Chemotherapy

A wide variety of chemotherapeutic agents may be used in accordance with the present embodiments. The term “chemotherapy” refers to the use of drugs to treat cancer. A “chemotherapeutic agent” is used to connote a compound or composition that is administered in the treatment of cancer. These agents or drugs are categorized by their mode of activity within a cell, for example, whether and at what stage they affect the cell cycle. Alternatively, an agent may be characterized based on its ability to directly cross-link DNA, to intercalate into DNA, or to induce chromosomal and mitotic aberrations by affecting nucleic acid synthesis.

Examples of chemotherapeutic agents include alkylating agents, such as thiotepa and cyclosphosphamide; alkyl sulfonates, such as busulfan, improsulfan, and piposulfan; aziridines, such as benzodopa, carboquone, meturedopa, and uredopa; ethylenimines and methylamelamines, including altretamine, triethylenemelamine, trietylenephosphoramide, triethiylenethiophosphoramide, and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards, such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, and uracil mustard; nitrosureas, such as carmustine, chlorozotocin, fotemustine, lomustine, nimustine, and ranimnustine; antibiotics, such as the enediyne antibiotics (e.g., calicheamicin, especially calicheamicin gammalI and calicheamicin omegaI1); dynemicin, including dynemicin A; bisphosphonates, such as clodronate; an esperamicin; as well as neocarzinostatin chromophore and related chromoprotein enediyne antiobiotic chromophores, aclacinomysins, actinomycin, authrarnycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins, such as mitomycin C, mycophenolic acid, nogalarnycin, olivomycins, peplomycin, potfiromycin, puromycin, quelamycin, rodorubicin, streptonigrin, streptozocin, tubercidin, ubenimex, zinostatin, and zorubicin; anti-metabolites, such as methotrexate and 5-fluorouracil (5-FU); folic acid analogues, such as denopterin, pteropterin, and trimetrexate; purine analogs, such as fludarabine, 6-mercaptopurine, thiamiprine, and thioguanine; pyrimidine analogs, such as ancitabine, azacitidine, 6-azauridine, carmofur, cytarabine, dideoxyuridine, doxifluridine, enocitabine, and floxuridine; androgens, such as calusterone, dromostanolone propionate, epitiostanol, mepitiostane, and testolactone; anti-adrenals, such as mitotane and trilostane; folic acid replenisher, such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids, such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSKpolysaccharide complex; razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; taxoids, e.g., paclitaxel and docetaxel gemcitabine; 6-thioguanine; mercaptopurine; platinum coordination complexes, such as cisplatin, oxaliplatin, and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-11); topoisomerase inhibitor RFS 2000; difluorometlhylornithine (DMFO); retinoids, such as retinoic acid; capecitabine; carboplatin, procarbazine,plicomycin, gemcitabien, navelbine, farnesyl-protein tansferase inhibitors, transplatinum, and pharmaceutically acceptable salts, acids, or derivatives of any of the above.

### 2. Radiotherapy

Other factors that cause DNA damage and have been used extensively include what are commonly known as γ-rays, X-rays, and/or the directed delivery of radioisotopes to tumor cells. Other forms of DNA damaging factors are also contemplated, such as microwaves, proton beam irradiation (U.S. Pat. Nos. 5,760,395 and 4,870,287), and UV-irradiation. It is most likely that all of these factors affect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

### 3. Immunotherapy

The skilled artisan will understand that additional immunotherapies may be used in combination or in conjunction with methods of the embodiments. In the context of cancer treatment, immunotherapeutics, generally, rely on the use of immune effector cells and molecules to target and destroy cancer cells. Rituximab (RITUXAN®) is such an example. The immune effector may be, for example, an antibody specific for some marker on the surface of a tumor cell. The antibody alone may serve as an effector of therapy or it may recruit other cells to actually affect cell killing. The antibody also may be conjugated to a drug or toxin (chemotherapeutic, radionuclide, ricin A chain, cholera toxin, pertussis toxin, etc.) and serve as a targeting agent. Alternatively, the effector may be a lymphocyte carrying a surface molecule that interacts, either directly or indirectly, with a tumor cell target. Various effector cells include cytotoxic T cells and NK cells

Antibody-drug conjugates have emerged as a breakthrough approach to the development of cancer therapeutics. Cancer is one of the leading causes of deaths in the world. Antibody-drug conjugates (ADCs) comprise monoclonal antibodies (MAbs) that are covalently linked to cell-killing drugs. This approach combines the high specificity of MAbs against their antigen targets with highly potent cytotoxic drugs, resulting in “armed” MAbs that deliver the payload (drug) to tumor cells with enriched levels of the antigen (Carter et al., 2008; Teicher 2014; Leal et al., 2014). Targeted delivery of the drug also minimizes its exposure in normal tissues, resulting in decreased toxicity and improved therapeutic index. The approval of two ADC drugs, ADCETRIS® (brentuximab vedotin) in 2011 and KADCYLA® (trastuzumab emtansine or T-DM1) in 2013 by FDA validated the approach. There are currently more than 30 ADC drug candidates in various stages of clinical trials for cancer treatment (Leal et al., 2014). As antibody engineering and linker-payload optimization are becoming more and more mature, the discovery and development of new ADCs are increasingly dependent on the identification and validation of new targets that are suitable to this approach (Teicher 2009) and the generation of targeting MAbs. Two criteria for ADC targets are upregulated/high levels of expression in tumor cells and robust internalization.

In one aspect of immunotherapy, the tumor cell must bear some marker that is amenable to targeting, i.e., is not present on the majority of other cells. Many tumor markers exist and any of these may be suitable for targeting in the context of the present embodiments. Common tumor markers include CD20, carcinoembryonic antigen, tyrosinase (p97), gp68, TAG-72, HMFG, Sialyl Lewis Antigen, MucA, MucB, PLAP, laminin receptor, erb B, and p155. An alternative aspect of immunotherapy is to combine anticancer effects with immune stimulatory effects. Immune stimulating molecules also exist including: cytokines, such as IL-2, IL-4, IL-12, GM-CSF, gamma-IFN, chemokines, such as MIP-1, MCP-1, IL-8, and growth factors, such as FLT3 ligand.

Examples of immunotherapies currently under investigation or in use are immune adjuvants, e.g., Mycobacterium bovis, Plasmodium falciparum, dinitrochlorobenzene, and aromatic compounds (U.S. Pat. Nos. 5,801,005 and 5,739,169; Hui and Hashimoto, 1998; Christodoulides et al., 1998); cytokine therapy, e.g., interferons α, β, and γ, IL-1, GM-CSF, and TNF (Bukowski et al., 1998; Davidson et al., 1998; Hellstrand et al., 1998); gene therapy, e.g., TNF, IL-1, IL-2, and p53 (Qin et al., 1998; Austin-Ward and Villaseca, 1998; U.S. Pat. Nos. 5,830,880 and 5,846,945); and monoclonal antibodies, e.g., anti-CD20, anti-ganglioside GM2, and anti-p185 (Hollander, 2012; Hanibuchi et al., 1998; U.S. Pat. No. 5,824,311). It is contemplated that one or more anti-cancer therapies may be employed with the antibody therapies described herein.

In some embodiments, the immunotherapy may be an immune checkpoint inhibitor. Immune checkpoints are molecules in the immune system that either turn up a signal (e.g., co-stimulatory molecules) or turn down a signal. Inhibitory checkpoint molecules that may be targeted by immune checkpoint blockade include adenosine A2A receptor (A2AR), B7-H3 (also known as CD276), B and T lymphocyte attenuator (BTLA), cytotoxic T-lymphocyte-associated protein 4 (CTLA-4, also known as CD152), indoleamine 2,3-dioxygenase (IDO), killer-cell immunoglobulin (KIR), lymphocyte activation gene-3 (LAG3), programmed death 1 (PD-1), T-cell immunoglobulin domain and mucin domain 3 (TIM-3) and V-domain Ig suppressor of T cell activation (VISTA). In particular, the immune checkpoint inhibitors target the PD-1 axis and/or CTLA-4.

The immune checkpoint inhibitors may be drugs such as small molecules, recombinant forms of ligand or receptors, or, in particular, are antibodies, such as human antibodies (e.g., International Patent Publication WO2015016718; Pardoll, Nat Rev Cancer, 12(4): 252-64, 2012; both incorporated herein by reference). Known inhibitors of the immune checkpoint proteins or analogs thereof may be used, in particular chimerized, humanized or human forms of antibodies may be used. As the skilled person will know, alternative and/or equivalent names may be in use for certain antibodies mentioned in the present disclosure. Such alternative and/or equivalent names are interchangeable in the context of the present invention. For example it is known that lambrolizumab is also known under the alternative and equivalent names MK-3475 and pembrolizumab.

In some embodiments, the PD-1 binding antagonist is a molecule that inhibits the binding of PD-1 to its ligand binding partners. In a specific aspect, the PD-1 ligand binding partners are PDL1 and/or PDL2. In another embodiment, a PDL1 binding antagonist is a molecule that inhibits the binding of PDL1 to its binding partners. In a specific aspect, PDL1 binding partners are PD-1 and/or B7-1. In another embodiment, the PDL2 binding antagonist is a molecule that inhibits the binding of PDL2 to its binding partners. In a specific aspect, a PDL2 binding partner is PD-1. The antagonist may be an antibody, an antigen binding fragment thereof, an immunoadhesin, a fusion protein, or oligopeptide. Exemplary antibodies are described in U.S. Pat. Nos. 8,735,553, 8,354,509, and 8,008,449, all incorporated herein by reference. Other PD-1 axis antagonists for use in the methods provided herein are known in the art such as described in U.S. Patent Application No. US20140294898, US2014022021, and US20110008369, all incorporated herein by reference.

In some embodiments, the PD-1 binding antagonist is an anti-PD-1 antibody (e.g., a human antibody, a humanized antibody, or a chimeric antibody). In some embodiments, the anti-PD-1 antibody is selected from the group consisting of nivolumab, pembrolizumab, and CT-011. In some embodiments, the PD-1 binding antagonist is an immunoadhesin (e.g., an immunoadhesin comprising an extracellular or PD-1 binding portion of PDL1 or PDL2 fused to a constant region (e.g., an Fc region of an immunoglobulin sequence). In some embodiments, the PD-1 binding antagonist is AMP-224. Nivolumab, also known as MDX-1106-04, MDX-1106, ONO-4538, BMS-936558, and OPDIVO©, is an anti-PD-1 antibody described in WO2006/121168. Pembrolizumab, also known as MK-3475, Merck 3475, lambrolizumab, KEYTRUDA©, and SCH-900475, is an anti-PD-1 antibody described in WO2009/114335. CT-011, also known as hBAT or hBAT-1, is an anti-PD-1 antibody described in WO2009/101611. AMP-224, also known as B7-DCIg, is a PDL2-Fc fusion soluble receptor described in WO2010/027827 and WO2011/066342.

Another immune checkpoint that can be targeted in the methods provided herein is the cytotoxic T-lymphocyte-associated protein 4 (CTLA-4), also known as CD152. The complete cDNA sequence of human CTLA-4 has the Genbank accession number L15006. CTLA-4 is found on the surface of T cells and acts as an “off” switch when bound to CD80 or CD86 on the surface of antigen-presenting cells. CTLA4 is a member of the immunoglobulin superfamily that is expressed on the surface of Helper T cells and transmits an inhibitory signal to T cells. CTLA4 is similar to the T-cell co-stimulatory protein, CD28, and both molecules bind to CD80 and CD86, also called B7-1 and B7-2 respectively, on antigen-presenting cells. CTLA4 transmits an inhibitory signal to T cells, whereas CD28 transmits a stimulatory signal. Intracellular CTLA4 is also found in regulatory T cells and may be important to their function. T cell activation through the T cell receptor and CD28 leads to increased expression of CTLA-4, an inhibitory receptor for B7 molecules.

In some embodiments, the immune checkpoint inhibitor is an anti-CTLA-4 antibody (e.g., a human antibody, a humanized antibody, or a chimeric antibody), an antigen binding fragment thereof, an immunoadhesin, a fusion protein, or oligopeptide.

Anti-human-CTLA-4 antibodies (or VH and/or VL domains derived therefrom) suitable for use in the present methods can be generated using methods well known in the art. Alternatively, art recognized anti-CTLA-4 antibodies can be used. For example, the anti-CTLA-4 antibodies disclosed in: U.S. Pat. No. 8,119,129, WO 01/14424, WO 98/42752; WO 00/37504 (CP675,206, also known as tremelimumab; formerly ticilimumab), U.S. Pat. No. 6,207,156; Hurwitz et al. (1998) Proc Natl Acad Sci USA 95(17): 10067-10071; Camacho et al. (2004) J Clin Oncology 22(145): Abstract No. 2505 (antibody CP-675206); and Mokyr et al. (1998) Cancer Res 58:5301-5304 can be used in the methods disclosed herein. The teachings of each of the aforementioned publications are hereby incorporated by reference. Antibodies that compete with any of these art-recognized antibodies for binding to CTLA-4 also can be used. For example, a humanized CTLA-4 antibody is described in International Patent Application No. WO2001014424, WO2000037504, and U.S. Pat. No. 8,017,114; all incorporated herein by reference.

An exemplary anti-CTLA-4 antibody is ipilimumab (also known as 10D1, MDX-010, MDX-101, and Yervoy®) or antigen binding fragments and variants thereof (see, e.g., WOO 1/14424). In other embodiments, the antibody comprises the heavy and light chain CDRs or VRs of ipilimumab. Accordingly, in one embodiment, the antibody comprises the CDR1, CDR2, and CDR3 domains of the VH region of ipilimumab, and the CDR1, CDR2 and CDR3 domains of the VL region of ipilimumab. In another embodiment, the antibody competes for binding with and/or binds to the same epitope on CTLA-4 as the above-mentioned antibodies. In another embodiment, the antibody has at least about 90% variable region amino acid sequence identity with the above-mentioned antibodies (e.g., at least about 90%, 95%, or 99% variable region identity with ipilimumab).

Other molecules for modulating CTLA-4 include CTLA-4 ligands and receptors such as described in U.S. Pat. Nos. 5,844,905, 5,885,796 and International Patent Application Nos. WO1995001994 and WO1998042752; all incorporated herein by reference, and immunoadhesions such as described in U.S. Pat. No. 8,329,867, incorporated herein by reference.

### 4. Surgery

Approximately 60% of persons with cancer will undergo surgery of some type, which includes preventative, diagnostic or staging, curative, and palliative surgery. Curative surgery includes resection in which all or part of cancerous tissue is physically removed, excised, and/or destroyed and may be used in conjunction with other therapies, such as the treatment of the present embodiments, chemotherapy, radiotherapy, hormonal therapy, gene therapy, immunotherapy, and/or alternative therapies. Tumor resection refers to physical removal of at least part of a tumor. In addition to tumor resection, treatment by surgery includes laser surgery, cryosurgery, electrosurgery, and microscopically-controlled surgery (Mohs' surgery).

Upon excision of part or all of cancerous cells, tissue, or tumor, a cavity may be formed in the body. Treatment may be accomplished by perfusion, direct injection, or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

### 5. Other Agents

It is contemplated that other agents may be used in combination with certain aspects of the present embodiments to improve the therapeutic efficacy of treatment. These additional agents include agents that affect the upregulation of cell surface receptors and GAP junctions, cytostatic and differentiation agents, inhibitors of cell adhesion, agents that increase the sensitivity of the hyperproliferative cells to apoptotic inducers, or other biological agents. Increases in intercellular signaling by elevating the number of GAP junctions would increase the anti-hyperproliferative effects on the neighboring hyperproliferative cell population. In other embodiments, cytostatic or differentiation agents can be used in combination with certain aspects of the present embodiments to improve the anti-hyperproliferative efficacy of the treatments. Inhibitors of cell adhesion are contemplated to improve the efficacy of the present embodiments. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with certain aspects of the present embodiments to improve the treatment efficacy.

### III. Kits

In various aspects of the embodiments, a kit is envisioned containing therapeutic agents and/or other therapeutic and delivery agents. In some embodiments, the present embodiments contemplates a kit for preparing and/or administering an antibody composition of the embodiments. The kit may comprise one or more sealed vials containing any of the pharmaceutical compositions of the present embodiments. The kit may include, for example, conjugated antibodies as well as reagents to prepare, formulate, and/or administer the components of the embodiments or perform one or more steps of the inventive methods. In some embodiments, the kit may also comprise a suitable container, which is a container that will not react with components of the kit, such as an eppendorf tube, an assay plate, a syringe, a bottle, or a tube. The container may be made from sterilizable materials such as plastic or glass.

The kit may further include an instruction sheet that outlines the procedural steps of the methods set forth herein, and will follow substantially the same procedures as described herein or are known to those of ordinary skill in the art. The instruction information may be in a computer readable media containing machine-readable instructions that, when executed using a computer, cause the display of a real or virtual procedure of delivering a pharmaceutically effective amount of a therapeutic agent.

### IV. Examples

The following examples are included to demonstrate preferred embodiments of the disclosure. It should be appreciated by those of skill in the art that the techniques disclosed in the examples which follow represent techniques discovered by the inventor to function well in the practice of the disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

### Example 1—Antibodies for Bone Metastasis

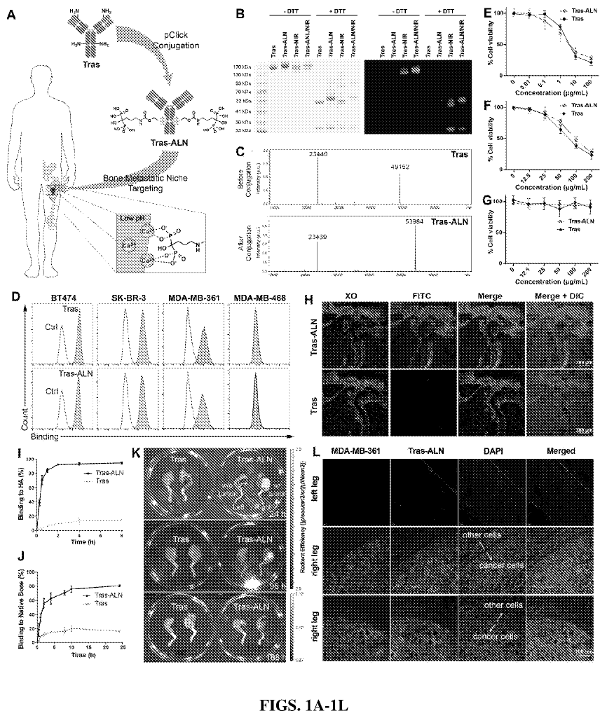

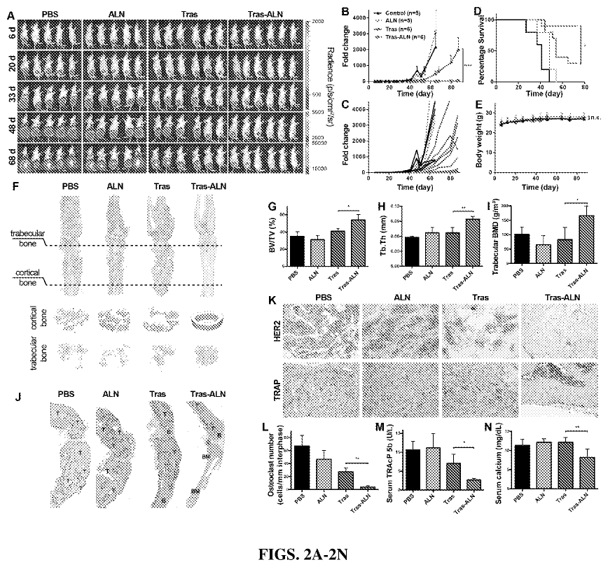

Development of the First Bone-Targeting Antibody using BonTarg. To explore the possibility of specifically delivering therapeutic antibodies to the bone via conjugation to BP molecules, a model was designed using the HER2 targeting antibody trastuzumab (Tras) and the BP drug Alendronate (ALN). ALN is a second-generation BP drug that is used as a bone-targeting agent as well as a regimen for treating osteoporosis and bone metastasis.³¹ To ensure that ALN conjugation does not impair the therapeutic efficacy of the antibody, a novel proximity-induced antibody conjugation strategy named pClick was employed.³¹ pClick technology enables the site-specific attachment of payloads to native antibodies under mild conditions, thus minimizing the disruption of binding to the antigen receptor or the FcγRIII receptor, the receptor responsible for activating antibody-dependent cell-mediated cytotoxicity (ADCC). The pClick technology does not rely on antibody engineering or on the UV/chemical/enzymatic treatments that characterize the generation of most therapeutic antibodies. To prepare trastuzumab-Alendronate conjugates (Tras-ALN), pClick was used to generate Tras containing an azide functional moiety, followed by reaction with bicyclo[6.1.0]nonyne (BCN)-functionalized ALN (FIG. 1A, and FIGS. 5-8). The resulting Tras-ALN was further purified on a desalting column and fully characterized by SDS-PAGE and ESI-MS (FIG. 1B, C). No unconjugated heavy chain or degradation products were revealed by SDS-PAGE, indicating a more than 95% coupling efficiency. ESI-MS analysis also revealed that more than 95% of the heavy chain was conjugated with the ALN molecule.

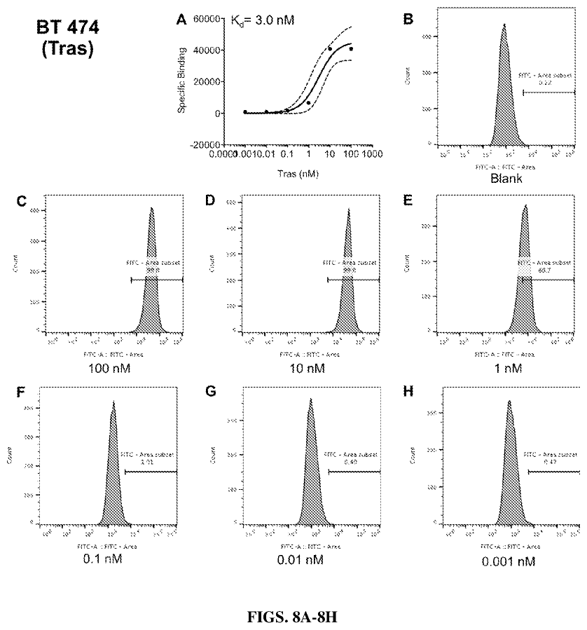

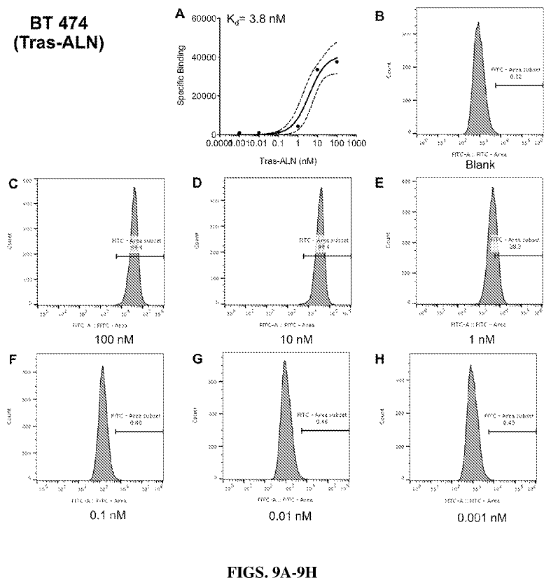

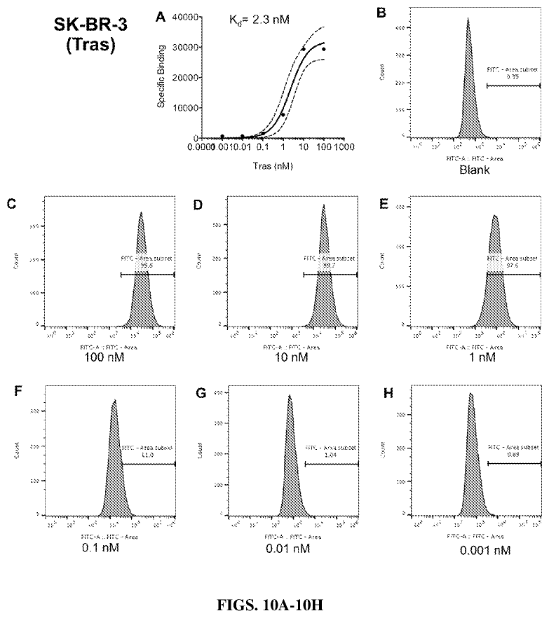

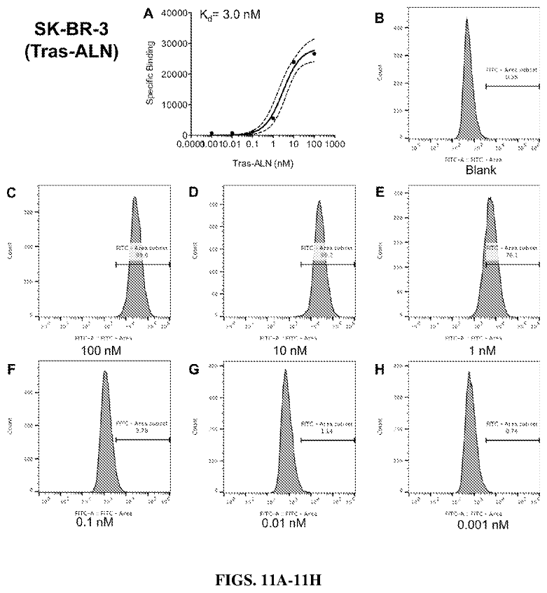

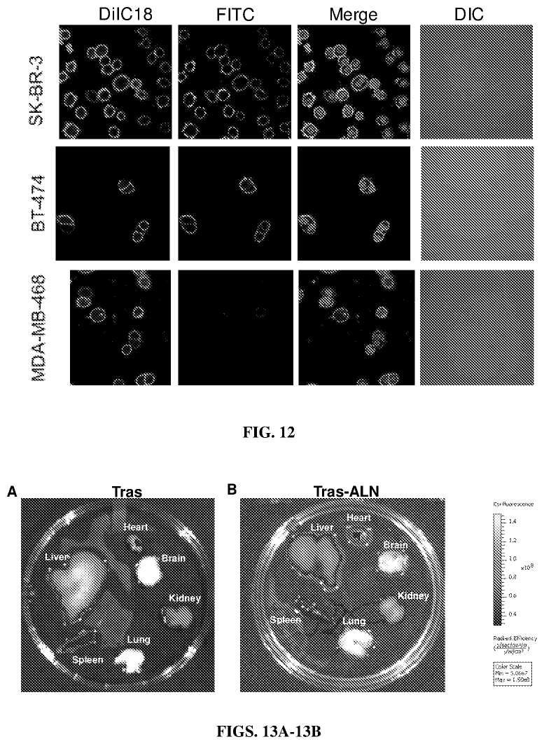

Antibody Conjugation to ALN Retains Antigen Binding and Specificity. To investigate the effect of ALN conjugation on antigen-binding affinity and specificity, binding affinities of Tras and Tras-ALN were assessed by flow cytometry analysis of HER2-positive and negative cell lines. FIG. 1D reveals that both Tras and Tras-ALN have strong binding affinities for the HER2-expressing cell lines BT474, SK-BR-3, and MDA-MB-361, but not for the HER2-negative cell line MDA-MB-468, suggesting that the antibody specificity was not altered by ALN conjugation (Table 1). The Kd values for binding to HER2-positive cells are within a similar range for Tras and Tras-ALN (BT474, 3.0 vs 3.8 nM; SK-BR-3, 2.3 vs 3.0 nM, respectively), indicating that ALN conjugation does not affect the strength of antigen-binding (FIGS. 8-11). Confocal fluorescent imaging further confirms that Tras-ALN retains antigen binding and specificity (FIG. 12). HER2-positive BT474 and SK-BR-3 cells, and HER2-negative MDA-MB-468 cells were incubated for 30 mins with fluorescein isothiocyanate (FITC)-labeled Tras-ALN. Confocal imaging indicates that cell-surface-associated fluorescence is only exhibited for HER2-positive BT474 and SK-BR-3 cells, and not for HER2-negative MDA-MB-468 cells (FIG. 12). Thus, ALN modification of Tras does not affect its antigen-binding affinity and specificity. Next, the Tras-ALN conjugate was tested for selective cytotoxicity against HER2-expressing and HER2-negative breast cancer cells. As shown in FIGS. 1E, 1F, 1G and Table 1, the Tras-ALN conjugate exhibits cytotoxic activity against HER2-positive BT-474 cells (EC₅₀ of 2.3±0.7 μg/ml) and MDA-MB-361 (EC₅₀ of 78+21 μg/ml) that is indistinguishable from that of Tras (EC₅₀ of 1.4±0.9 μg/ml and EC₅₀ of 57±10 μg/ml). Neither antibody kills HER2-negative MDA-MB-468 cells (EC₅₀>500 μg/ml). These results indicate that conjugation of the negatively charged moiety ALN preserves the antigen-binding and in vitro anti-tumor cell activity of the Tras antibody.

Enhanced Targeting of the Bone Metastatic Niche by Tras-ALN in vitro and in vivo. Next, the ability of the Tras-ALN conjugate to target bone tissue was explored. Non-decalcified bone sections from C57BL/6 mice were incubated overnight at 4° C. with 50 μg/mL Tras or Tras-ALN conjugate, followed by labeling with FITC-labeled anti-human IgG. Before imaging via confocal laser scanning microscopy, these bone sections were further stained for 30 min with 4 μg/mL xylenol orange (XO, known to label bone). A FITC signal was observed in sections stained with the Tras-ALN conjugate, but not in sections stained with unmodified Tras (FIG. 1H). Furthermore, localization of the Tras-ALN signal correlated well with the XO signal, confirming the specific targeting of bone by Tras-ALN. To quantify the difference in affinity between binding of the Tras-ALN conjugate and unmodified Tras, Tras-ALN and Tras were incubated with hydroxyapatite or native bone. As shown in FIGS. 11 and 1J, unmodified Tras exhibited only slight binding to HA or native bone. Even with an increase in the incubation time, the binding affinity of Tras did not change significantly. In contrast, approximately 80%-90% of Tras-ALN was bound to HA and native bone after 2 h and 10 h, respectively.

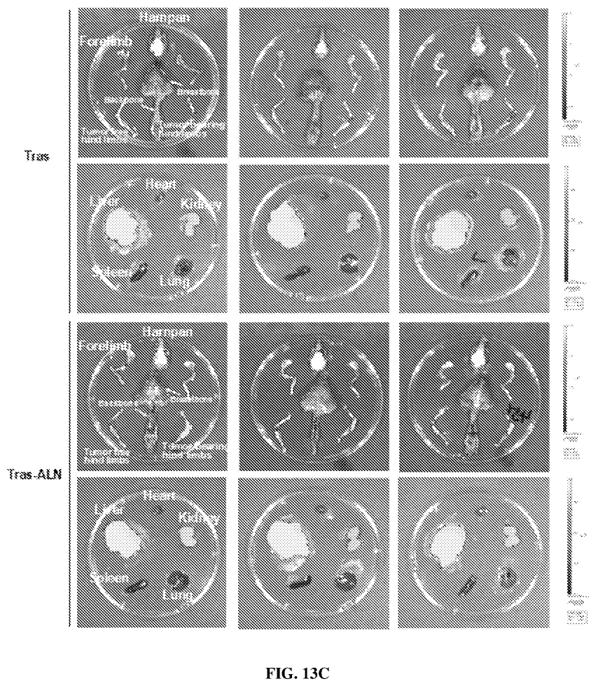

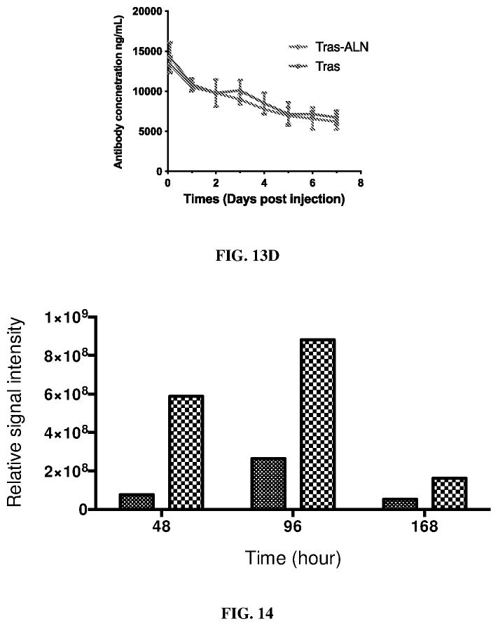

Encouraged by the in vitro bone-targeting ability of ALN-conjugated Tras, an in vivo biodistribution study was carried out with the Tras-ALN conjugate using a tumor xenograft model. To facilitate the detection of antibodies in vivo, Tras and Tras-ALN were first conjugated with Cyanine 7.5 (Cy7.5)-hydroxysuccinimide (NHS) ester. The resulting Cy7.5 labeled conjugates were analyzed using SDS-PAGE. As expected, fluorescence was associated only with the Cy7.5-labeled conjugates (FIG. 1). An important feature of BP is that uptake of bisphosphonate into bone metastases is much higher than in healthy bone tissue, due to the relatively low pH of the bone metastatic microenvironment.³²⁻³⁵ To investigate if ALN-Tras can specifically target bone metastases, thus minimizing on-target toxicity to normal bone tissue, the targeting properties of ALN-Tras was evaluated in a bone tumor model. A bone micrometastasis model was created by using intra-iliac artery (IIA) injection of MDA-MB-361 cells labeled with luciferase and red fluorescent protein (RFP) into the right hind limbs of nude mice. IIA injection is a novel technology recently developed in for establishing bone micrometastases. The method allows for selective delivery of cancer cells into hind limb bones without causing tissue damage.³⁶⁻³⁸ This technology allows sufficient time for some indolent cells to eventually colonize the bone as well as a large number of cancer cells to specifically colonize the bone, thereby enriching micrometastases in early stages. This allows for swift detection and robust quantification of micrometastases. Establishment of micrometastases was followed by treatment with Tras or Tras-ALN (1 mg/kg). 24, 96 or 168 hrs after administration of antibody or antibody conjugate, the major organs, including heart, liver, spleen, kidney, lung, and bone, were removed and analyzed using the Caliper IVIS Lumina II imager (FIGS. 1K and 13). Significantly, ex vivo fluorescence images at 96 h post-injection of antibody confirmed clear accumulation of Cy7.5-labeled Tras-ALN in the bone compared with Cy7.5-labeled Tras (FIGS. 1K and 14). Furthermore, the uptake of Tras-ALN into cancer-bearing bones was significantly higher than into healthy bone tissue. This is consistent with previous observations that BP molecules prefer to bind to the bone matrix in an acidic tumor environment.³⁹ In a separate study, unlabeled Tras-ALN (1 mg/kg) was administered into the nude mice bearing MDA-MB-361 tumor in the right hind limb. Bone sections from this study were also stained with FITC-labeled anti-human IgG, RFP and DAPI. FITC signals were only observed in sections from the right leg harboring MDA-MB-361 tumors. No FITC signals were detected in the left leg without tumors (FIG. 1L). Significantly, the FITC signal correlated well with the red fluorescence of MDA-MB-361 cells, suggesting that Tras-ALN conjugate selectively targets the bone metastatic site, but not healthy bone. These results demonstrate that ALN conjugation can significantly enhance the delivery and concentration of therapeutic antibodies in bone metastatic sites.

Next, the effect of ALN-conjugation on the pharmacokinetics and FcRn binding of antibodies was evaluated. A single dose of 1 mg/kg Tras and Tras-ALN in PBS were injected retro-orbitally, and serum was collected at regular intervals for 7 days and analyzed by Trastuzumab ELISA Kit. The serum concentration of both Tras and Tras-ALN decreased and did not show significant differences (FIG. 13). Next, the effect of ALN conjugation on FcRn binding was determined. It was found that the ALN conjugation doesn't have an obvious effect on the FcRn binding at pH 6.0 (Table 4).

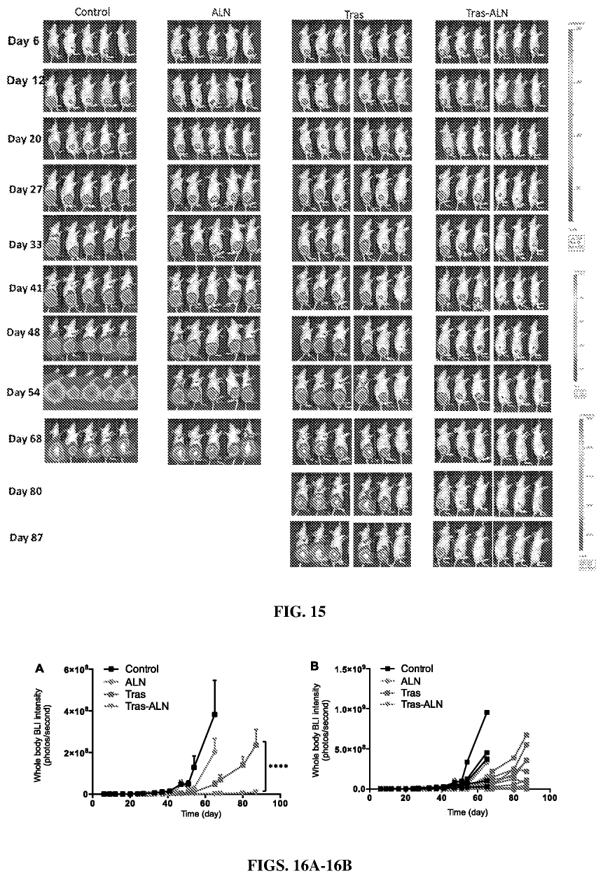

Enhanced Therapeutic Efficacy of Tras-ALN Against Bone Micrometastases. To determine whether bone-targeting trastuzumab represents a novel therapeutic approach for treating micrometastases of breast cancer in the bone, a xenograft study was carried out in nude mice. Using intra-iliac artery (IIA) injection, the right hind limbs of nude mice were inoculated with 5×10⁵ MDA-MB-361 cells labeled with firefly luciferase. Five days after the IIA injections, mice were treated with phosphate-buffered saline (PBS), ALN (10 μg/kg), Tras (1 mg/kg), or Tras-ALN (1 mg/kg) via retro-orbital injection. As shown in FIGS. 2A and 15, micrometastases in PBS- and ALN-treated mice accumulated rapidly, while development of lesions in Tras- and Tras-ALN-treated mice was delayed. Whole-body bioluminescence imaging (BLI) signals suggested that treatment with Tras-ALN resulted in more significant inhibition of micrometastasis progression, compared to that seen in Tras-treated mice (FIGS. 16A and 16B). The increases in BLI from day 6 to 87 showed that the Tras-ALN-treated group had fewer fold-increases in the tumor sizes compared to Tras-treated group (Tras vs Tras-ALN: 1965.1±798.3 vs 42.6±23.4, FIGS. 2B and 2C). As the bone metastasis were built in the hind limbs, the effect of Tras-ALN on the BLI signal in the hind limbs was also quantified. Similar to whole-body BLI signal, Tras-ALN-treated group had less BLI signal intensity and fewer fold-increase in the hind limbs (FIG. 17). Moreover, survival of Tras-ALN-treated mice was notably enhanced compared to that of PBS-, ALN-, and Tras-treated mice, demonstrating the efficacy of Tras-ALN against HER2-positive cells in vivo (FIG. 2D). Furthermore, no weight loss as a sign of ill health was observed in any of the treated mice, suggesting the absence of toxicity associated with the bone-targeting antibodies (FIG. 2E).

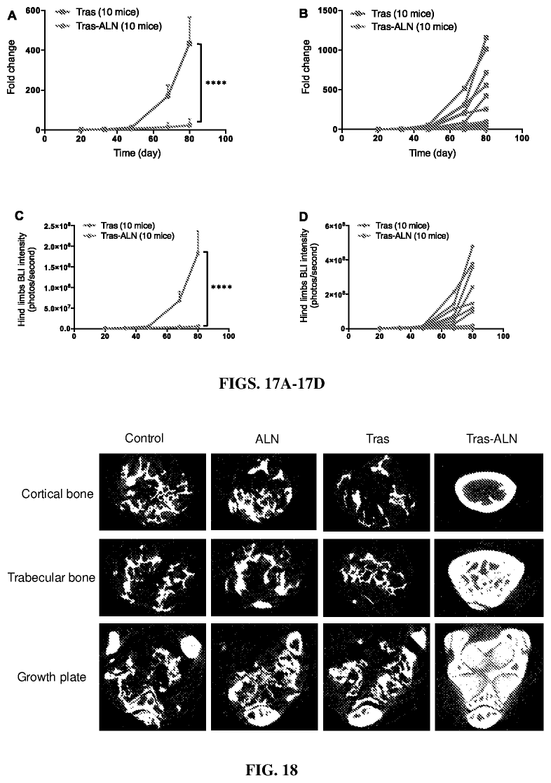

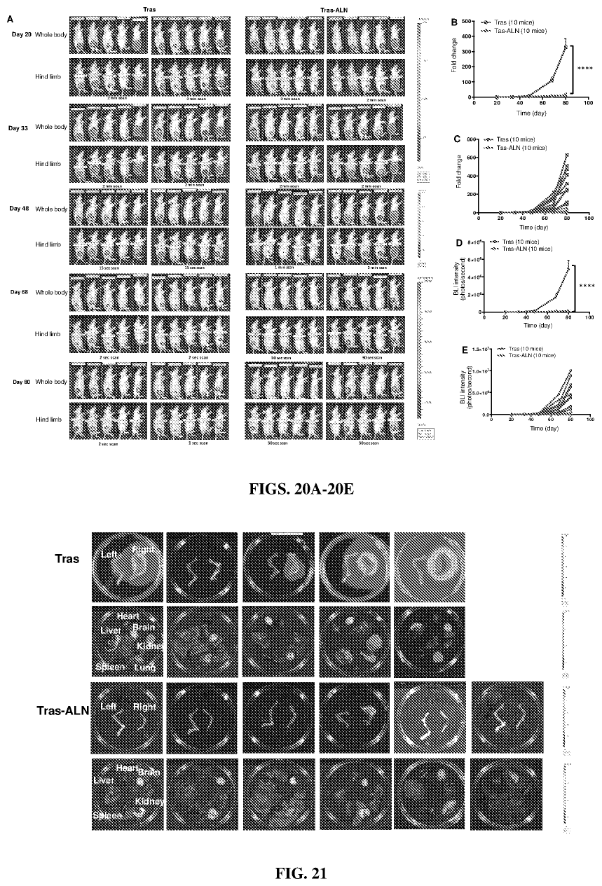

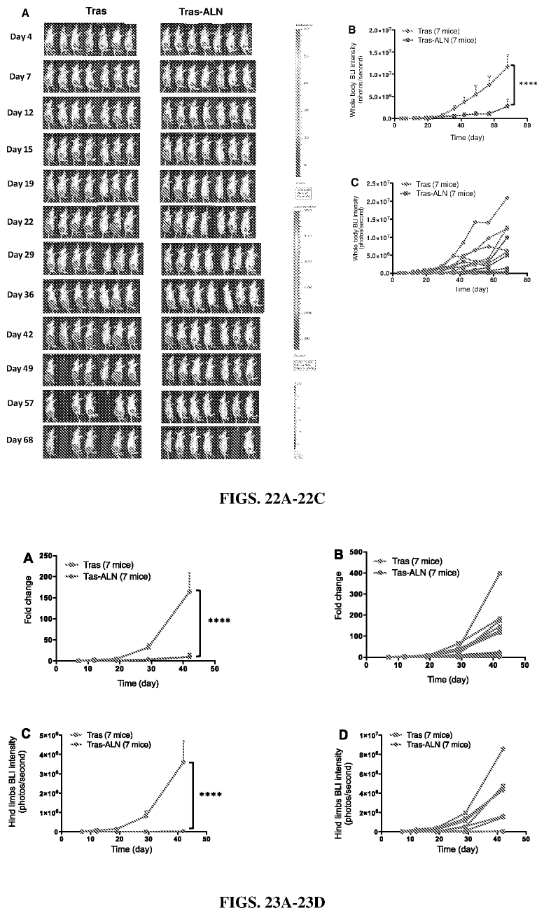

These results were further confirmed by micro-computed tomography (microCT) data and histology, emphasizing the finding that bone-targeting antibodies can decrease both the number and the extent of osteolytic lesions. As shown in FIGS. 2F and 18, femurs from PBS-, ALN-, and Tras-treated groups exhibited significant losses of bone mass, while bone loss in the Tras-ALN-treated group was much reduced. Quantitative analysis revealed that the Tras-ALN-treated group had significantly higher bone volume (FIG. 2G, 6B: BV/TV (%), 35.08±2.65 vs 56.67±1.02, p=0.0005) thicker trabecular bone (FIG. 2H, Tb.Th (mm), 0.061±0.003 vs 0.094±0.002, p=0.003), and higher trabecular bone mineral density (FIG. 21, BMD (mg/mm³), 101.16±12.24 vs 165.94±12.84, p=0.035) compared to the Tras-treated group.

Tumor size was also analyzed by histomorphometric analysis of the bone sections. Tibiae and femurs from the PBS-treated and ALN-treated groups had high tumor burdens (FIG. 2J). Tras treatment slightly reduced the tumor burden, but the reduction was not statistically significant. In contrast, a significant reduction of tumor burden was observed in the Tras-ALN-treated group. Histological examination of the bone samples from various treatment groups reveals that bone matrix is generally destroyed in bones with high tumor burden, whereas bones with less tumor burden in the Tras-ALN-treated group exhibit intact bone matrix. The reduction of tumor burden was also confirmed by HER2 immunohistochemistry (IHC). As shown in FIG. 2K, the number of HER2-positive breast cancer cells was dramatically decreased in Tras-ALN-treated mice, even though HER2 expression by individual tumor cells was unchanged. This suggests that extended treatment with Tras-ALN has no effect on HER2 expression by MDA-MB-361 cells.

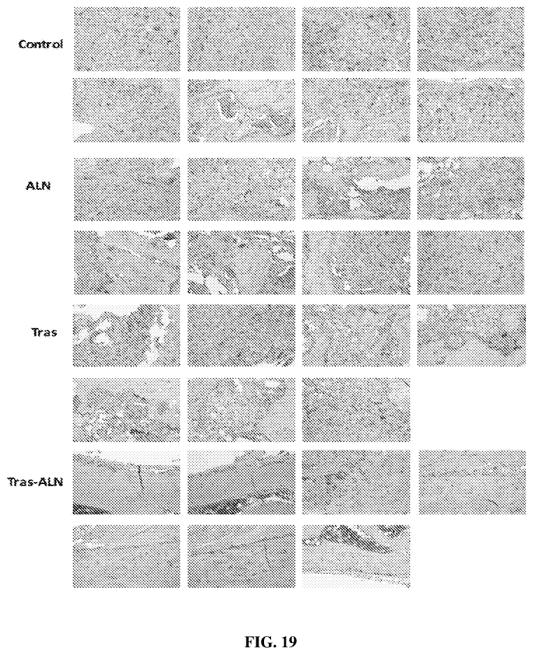

To examine Tras-ALN inhibition of tumor-induced osteolytic bone destruction, the bone-resorbing, tartrate-resistant, acid phosphatase-positive multinucleated osteoclasts were examined in bone samples (FIG. 2K). Tartrate-resistant acid phosphatase (TRAP) staining identified reduced numbers of osteoclasts (pink cells) lining the eroded bone surface in Tras-ALN-treated mice, compared to Tras-treated mice (FIGS. 2K, 2L, and 19). Serum TRAcP 5b and calcium levels, indicators of bone resorption, were also measured at the experimental endpoint. Significantly higher reductions in bone resorption were observed in the Tras-ALN-treated group (FIGS. 2M and 2N). Taken together, these results indicate that bisphosphonate modification of therapeutic antibodies significantly enhanced their ability to retard the development of micrometastasis-induced osteolytic lesions (Table 2).

To further evaluate the therapeutic efficacy of Tras-ALN in the presence of both primary and secondary tumors, a xenograft study was carried out in nude mice, using both mammary fat pad and IIA injections. For the cells inoculated in the right hind limbs, luciferase-labeled MDA-MB-361 cells (2×10⁵) were used. For the mammary fat pad injection, non-labeled MDA-MB-361 cells (1×10⁶) were injected. Six days after injection, mice were treated with Tras (1 mg/kg) and Tras-ALN (1 mg/kg). The tumor progressions of primary and bone metastasis were monitored by tumor size measurement and bioluminescence, respectively. Compared with the Tras-treated group, Tras-ALN had a significant effect in preventing tumor growth in the hind limb (FIG. 27A-B). However, there was no significant growth difference for the mammary fat pad tumor (FIG. 27C). These results suggested that Tras-ALN has a better therapeutic effect on bone metastases, but a similar effect on primary tumor compared with wild type Tras.

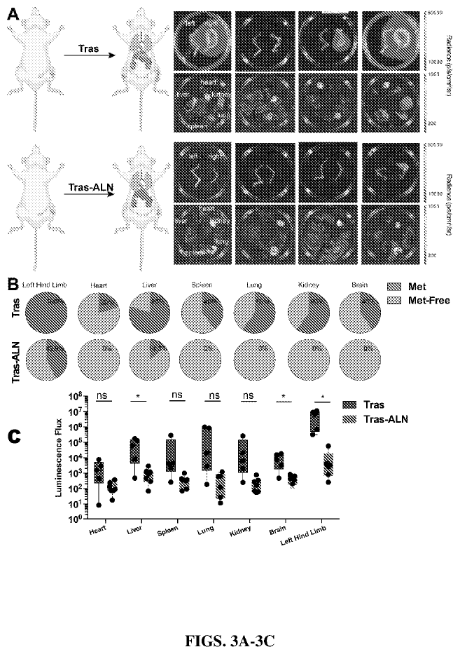

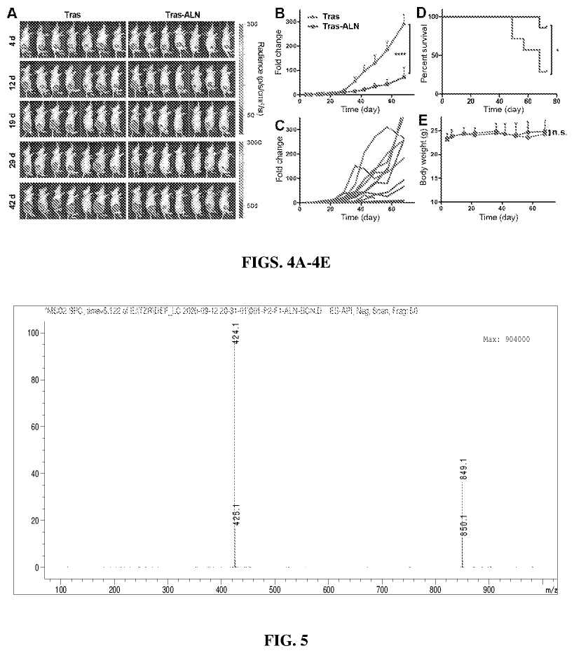

Tras-ALN inhibits multi-organ metastases from bone lesions. In more than two-thirds of cases, bone metastases are not confined to the skeleton, but rather give rise to subsequent metastases to other organs.^{9,10} While IIA injection was used to investigate early-stage bone colonization, as these bone lesions progress over an 8-12 week period, metastases begin to appear in other organs, including additional bones, lungs, liver, kidney, and brain. Hence, the ability of Tras-ALN to reduce the metastasis of HER2-positive MDA-MB-361 cancer cells to other organs was investigated. As before, 5×10⁵ MDA-MB-361 cells labeled with firefly luciferase were introduced into the right hind limbs of nude mice via IIA injection, followed by treatment with Tras (1 mg/kg) and Tras-ALN (1 mg/kg). Then, mice were subjected to whole-body BLI twice a week following tumor-cell injection. The whole-body and hind limbs BLI signals were quantified and showed in FIG. 20A. Secondary metastases in various organs were calculated as follows: BLI signal in whole body—BLI signal in hind limbs. As shown in FIG. 20, There was a time-dependent increase in the organs BLI signal to 10⁶ photons sec⁻¹ in the Tras treated group. And there was significant inhibition of BLI signal accumulation in organs of Tras-ALN-treated group (P<0.0001). At the endpoint of the study, mice were euthanized, and organs were harvested for bioluminescence imaging. Much higher levels of right hind limb (100%), heart (20%), liver (80%), spleen (40%), lung (60%), kidney (60%) and brain metastasis (40%) were observed in the Tras treated group, compared to the right hind limb (42.9%) and liver (14.3%, FIGS. 3A, 3B, and 21) in the Tras-ALN group. Other organs such as the lungs, spleen, kidney, and brain were devoid of metastases in Tras-ALN-treated mice. Our data indicated that bone-targeting antibodies, compared to unmodified antibodies, can significantly inhibit multi-organ metastases resulting from the dissemination of initial bone micrometastases. Mice treated with Tras-ALN exhibited fewer metastases to other organs than mice in the other treatment groups, establishing the ability of bone-targeting antibodies to inhibit “metastasis-to-metastasis seeding”.

TABLE 1
Potency and cell-surface reactivity of Tras and Tras-ALN
against breast cancer epithelial cell lines.

| | Her2 | IC50 (μg/mL) | | MFI increase (fold) | |
|---|---|---|---|---|---|
| Cell Line | Expression | Trash | Tras-ALN | Tras | Tras-ALN |
| BT-474 | 3+ | 1.4 ± 0.9 | 2.3 ± 0.7 | 47.57 | 43.72 |
| SK-BR-3 | 3+ | | | 57.01 | 51.70 |
| MDA-MB-361 | 2+ | 57 ± 10 | 78 ± 21 | 23.50 | 31.30 |
| MDA-MB-468 | 0 | >500 | >500 | 1.01 | 1.06 |

Abbreviations: MFI, median fluorescence intensity. Binding was determined as the mean fold increase in median fluorescence over the PBS control.

TABLE 2
Comparison of different treatment groups in multiple assays (MDA-MB-361 model).

| | | | Osteoclast activity | | | | |
|---|---|---|---|---|---|---|---|
| | BLI | | Osteoclast | Serum TRACP | Serum | Body weight | |
| Treatment | Progression^{a} | Fold-increase^{b} | number^{c} | 5b^{d} | Calcium^{e} | Progression^{f} | Survival^{g} |
| PBS vs ALN | NS | NS | * | NS | NS | NS | NS |
| PBS vs Tras | **** | **** | **** | NS | NS | NS | NS |
| PBS vs Tras-ALN | **** | **** | **** | *** | ** | NS | *** |
| ALN vs Tras | **** | **** | * | NS | NS | NS | NS |
| ALN vs Tras-ALN | **** | **** | **** | *** | * | NS | *** |
| Tras vs Tras-ALN | **** | **** | ** | * | ** | NS | * |

Abbreviations: ANOVA, analysis of variance; BLI, bioluminescence imaging; TRAP, tartrate-resistant acid phosphatase.
ªSignal intensity of BLI in whole body over the course of the experiment.
^{b}Signal intensity fold-increase of BLI after treatment (BLI of day 87/BLI of day 6).
^{c}Osteoclast number measurement from TRAP-stained tibia/femur sections at the end of experiment.
^{d}Serum TRACP 5b concentration at the end of experiment.
^{e}Serum calcium concentration at the end of experiment.
^{f}Body weight progression over the course of the experiment.
^{g}The mice BLI intensity over 10⁷ was considered to reach the endpoint.
^{a,b}were analyzed statistically by using a two-way repeated-measure ANOVA followed by Sidak's multiple comparisons test.
^{c,d,e,f}were analyzed by using a one-way ANOVA followed by Tukey's multiple comparisons test.
^{g}was analyzed by using a log-rank test.
**** P < 0.0001,
*** P < 0.001,
** P < 0.01,
* P < 0.05,
NS represents P > 0.05.

TABLE 3
Comparison of Tras and Tras-ALN groups in multiple assays (MCF 7 model).

| | | | Osteoclast activity | | | |
|---|---|---|---|---|---|---|
| | BLI | | Serum | Serum | Body weight | |
| Treatment | Progressionª | Fold-increase^{b} | Calcium^{c} | TRACP 5b^{d} | Increase^{e} | Survival^{f} |
| Tras vs Tras-ALN | **** | **** | * | * | NS | * |

Abbreviations: ANOVA, analysis of variance; BLI, bioluminescence imaging; TRAP, tartrate-resistant acid phosphatase.
ªSignal intensity of BLI in whole body over the course of the experiment.
^{b}Signal intensity fold-increase of BLI after treatment (BLI of day 68/BLI of day 6).
^{c}Serum calcium concentration at the end of experiment.
^{d}Serum TRACP 5b concentration at the end of experiment.
^{e}Body weight progression over the course of the experiment.
^{f}The mice BLI intensity over 107 was considered to reach the endpoint.
^{a,b}were analyzed statistically by using a two-way repeated-measure ANOVA followed by Sidak's multiple comparisons test.
^{c,d,e}were analyzed by using a one-way ANOVA followed by Tukey's multiple comparisons test.
^{f}was analyzed by using a log-rank test.
**** P < 0.0001,
* P < 0.05,
NS represents P > 0.05.

TABLE 4
Binding to Human FcRn at pH = 6.

| Antibodies | K_{D} (nM) |
|---|---|
| Tras | 33.3 ± 13.6 |
| Tras-ALN | 39.1 ± 14.8 |

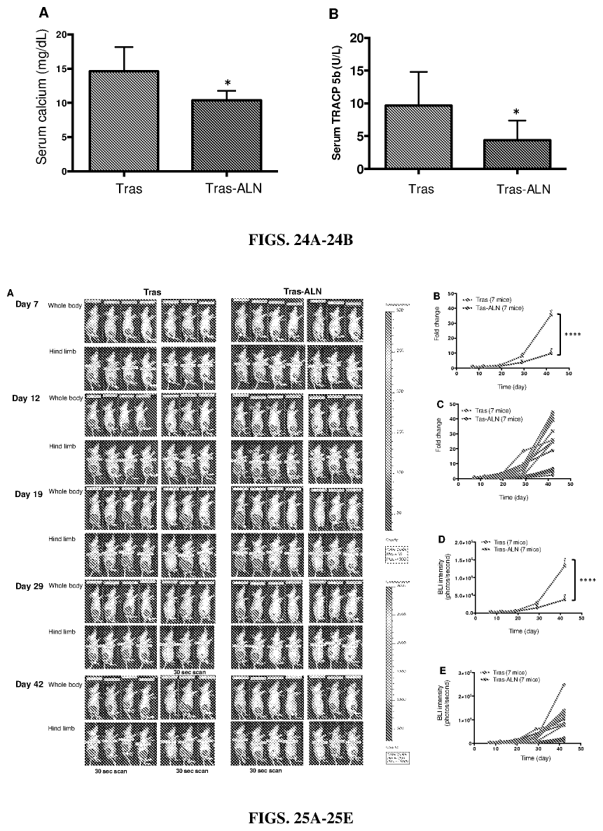

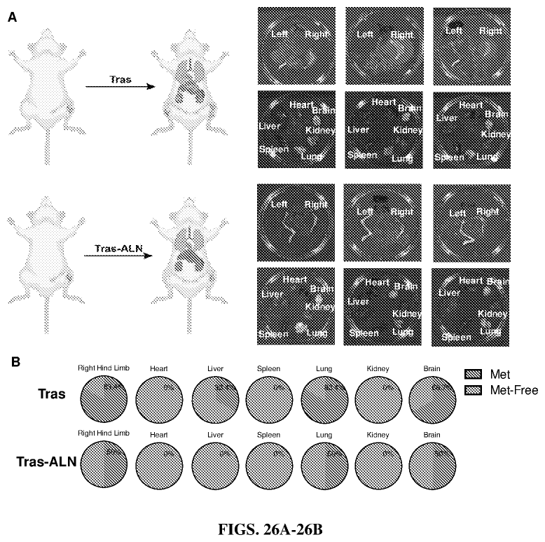

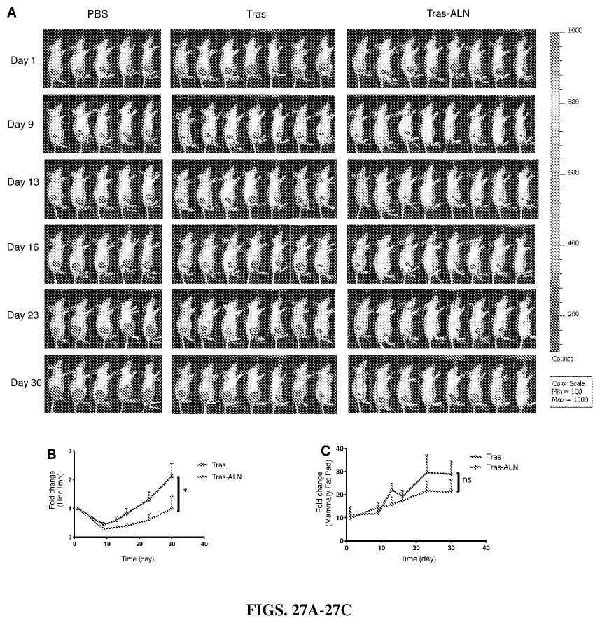

Enhanced Therapeutic Efficacy of Tras-ALN in a HER2-negative model. Previous reports indicate that a substantial portion of the minimal residual disease seen in HER2-negative patients may nevertheless be due to HER2 signaling^{40,41}. It was also reported that HER2 signaling may mediate stem cell properties in a subpopulation of HER2-negative cells, this raises the possibility that anti-HER2 treatment may be able to eradicate bone metastases of both HER2-positive and negative breast cancer.⁴² The therapeutic effects of Tras-ALN was therefore evaluated using breast cancer cells that are not HER2-positive but exhibit HER2 up-regulation specifically in bones. Intra-iliac artery (IIA) injection was used to deliver MCF-7 (HER2−, ER+) cancer cells into hind limb bones,^{36,38} followed by treatment with Tras or Tras-ALN (7 mice per group, 1 mg/kg). Mice were imaged twice a week and signal intensity of whole-body and hind limbs and were quantified. As shown in FIGS. 4, 22 and 23, treatment with Tras-ALN resulted in more significant inhibition of tumor growth than seen in Tras-treated mice, demonstrating the efficacy of Tras-ALN against HER2-negative cells in vivo (p<0.005). Meanwhile, significant reductions of serum TRACP 5b (4.41±1.12 U/L, p<0.05) and serum calcium (10.36±0.53 mg/dL, p<0.05) levels were observed in Tras-ALN-treated group (FIG. 24). Similar to HER2+ model, secondary metastases in various organs were also exhibited significant reductions in BLI signal (P<0.0001) over the course of the study (FIG. 25). Next, the ability of Tras-ALN to inhibit multi-organ metastases from bone lesions was also evaluated ex vivo. At day 68, metastatic cells were observed in the right hind limb (83.4%), liver (33.4%), lung (83.4%), and brain (66.7%) in the Tras-treated group, compared to values found in the right hind limb (50%), lung (50%) and brain (50%, FIG. 26) of Tras-ALN treated mice. These data suggest that the bone-targeting Tras-ALN conjugate may be useful in preventing the progression of HER2-negative bone micrometastases to overt bone metastases, as well as blocking the secondary metastasis of HER2-negative cells to other organs (Table 3).

Thus, it was shown that conjugation of bone-targeting moieties can be used to develop an innovative bone targeting (BonTarg) technology that enables the preparation of antibodies with both antigen and bone specificity. The data suggest that modification of the therapeutic HER2 antibody trastuzumab (Tras) with the bone-targeting bisphosphonate molecule, Alendronate (ALN), results in enhanced conjugate localization within the bone metastatic niche, relative to other tissues, raising the intriguing possibility that the bone-targeting antibody represents an enhanced targeted therapy for patients with bone metastases. The affinity of ALN for bone tissue helps overcome physical and biological barriers in the bone microenvironment that impede delivery of therapeutic antibodies, thereby enriching and retaining Tras in the bone. The Tras-ALN conjugate can reach higher concentrations in the bone metastatic niche, relative to healthy bone tissues, due to the low pH of bone tumor sites.”

Via its use of site-specific modification with bone-targeting moieties, BonTarg technology represents an innovative platform for specific delivery of therapeutic antibodies to the bone metastatic site. The resulting bone-targeting antibodies exhibit improved in vivo therapeutic efficacy in the treatment of breast cancer micrometastasis and in the prevention of secondary metastatic dissemination from the initial bone lesions. This type of precision delivery of biological medicines to the bone niche represents a promising avenue for treating bone-related diseases. The enhanced therapeutic profile of our bone-targeted HER2 antibody in treating microscopic BCa bone metastases will inform the extension of BonTarg strategies to treatment of other metastatic cancers and bone diseases.

### Example 2—Materials and Methods

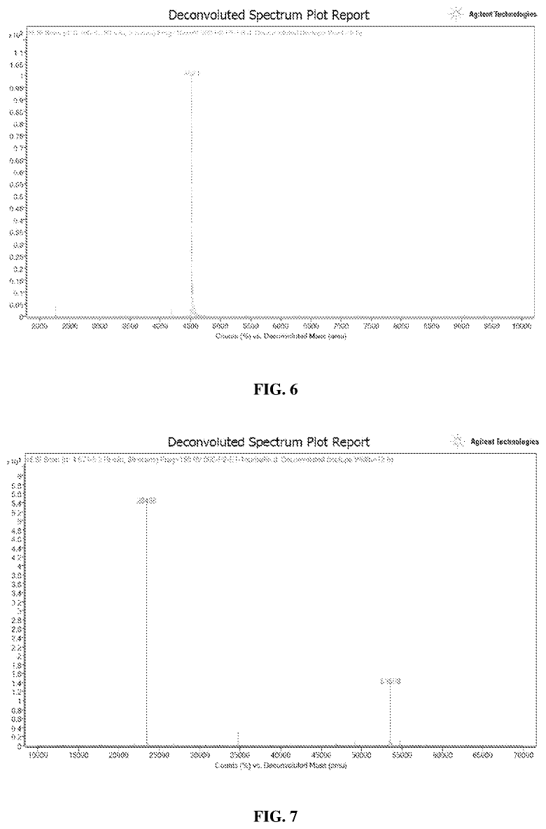

Construction of Tras-ALN conjugates. The non-canonical amino acid azide-Lys was incorporated at the C terminus of the ssFB-FPheK peptide via solid-phase peptide synthesis (FIG. 6). After HPLC purification, the peptide was denatured with 6 M urea and stepwise dialyzed to remove urea and allow peptide refolding. After buffer-exchange into PBS (pH 8.5), 32 equiv of ssFB-azide peptide was co-incubated with Tras (BS046D from Syd labs) in PBS (pH 8.5) buffer at 37° C. for two days. The Tras-azide conjugate was then purified via a PD-10 desalting column to remove excess ssFB-azide. The Tras-azide conjugate was characterized by ESI-MS. ESI-MS: expect 53564, found: 53558 (FIG. 7). 10 equiv of BCN-ALN was added to the solution at RT over night to selectively react with the azide group on the conjugate. Finally, the ALN labelled antibody conjugate was purified via a PD-10 desalting column to remove excess ALN-BCN. The conjugate was characterized by ESI-MS. ESI-MS: expect 53988, found: 53984 (FIG. 1C).

Cell lines. MDA-MB-361, MCF-7, BT474, SK-BR-3, and MDA-MB-468 cell lines were cultured according to ATCC instructions. Firefly luciferase and GFP labelled MDA-MB-361 and MCF 7 cell lines were generated as previously described.⁵¹

HA binding assay. Briefly, Tras or ALN-Tras was diluted in 1 mL PBS in an Eppendorf tube. Hydroxyapatite (15 equiv, 15 mg) was added, and the resulting suspension was shaken at 220 rpm at 37° C. Samples without hydroxyapatite were used as controls. After 0.25, 0.5, 1, 2, 4 and 8 hours, the suspension was centrifuged (3000 rpm, 3 min) and the absorbance of the supernatant at 280 nm was measured by Nanodrop. The percent binding to HA was calculated as follow, where OD represents optical density:

$$[ ( {OD}_{without HA} - {OD}_{with HA} ) / ( {OD}_{without HA} ) ] \times 100 \% .$$

Native bone binding assay. Long bones of mice were cut into small fragments, washed with distilled H₂O and anhydrous ethanol, and then dried at room temperature overnight. For binding studies, Tras or ALN-Tras was diluted in 1 mL PBS in an Eppendorf tube. 30 mg dried bone fragments were added to the tube, and the resulting suspension was shaken at 220 rpm at 37° C. Samples without bone fragments were used as controls. After 0.25, 0.5, 1.0, 2.0, 4.0 and 8.0 h, the suspensions were centrifuged (3000 rpm, 5 min) and the absorbance at 280 nm of the supernatant was measured by Nanodrop. The percent binding to native bone was calculated according to the following formula, where OD represents optical density:

$$[ ( {OD}_{without native bone} - {OD}_{with native bone} ) / ( {OD}_{without native bone} ) ] \times 100 \% .$$

In vitro cytotoxicity of Tras and Tras-ALN. SK-BR-3, BT474, and MDA-MB-468 cells were seeded in 200 μL of culture medium into 96-well plates at a density of 2×10³ cells/well and incubated overnight to allow attachment. Culture medium was then removed, replaced by different concentrations of Tras and Tras-ALN dissolved in culture medium, and then incubated for 4 d. 20 μL of MTT solution (5 mg/mL) was then added to each well and incubated for another 4 h. Medium was aspirated and 150 μL DMSO was added to each well. The absorbance at 570 nm was measured by microplate reader (Infinite M Plex by Tecan) to quantify living cells.

Flow cytometry. Cancer cells (3×10³) were re-suspended in 96-well plates and stained with 30 μg/mL Tras and Tras-ALN for 30 min at 4° C. After staining, cells were washed twice with PBS and then further incubated with Fluorescein (FITC) AffiniPure Goat Anti-Human IgG (H+L) (code: 109-095-003, Jackson Immunology) for 30 min at 4° C. Fluorescence intensity was determined using a BD FACSVerse (BD Biosciences).

Determination of Kd values. The functional affinity of Tras-ALN for HER2 was determined as reported.⁵² Briefly, 2×10⁵ SK-BR-3, BT474, MDA-MB-361, or MDA-MB-468 cells were incubated with increasing concentrations of Tra and Tras-ALN for 4 hours on ice. After washing away unbound material, bound antibody was detected using Fluorescein (FITC) AffiniPure Goat Anti-Human IgG (H+L) (Jackson Immunology). Cells were analyzed for fluorescence intensity after propidium iodide (Molecular Probes, Eugene, OR) staining. The linear portion of the saturation curve was used to calculate the dissociation constant, KD, using the Lineweaver-Burk method of plotting the inverse of the median fluorescence as a function of the inverse of the antibody concentration. The KD was determined as follows: 1/F=1/Fmax+(KD/Fmax)(1/[Ab]), where F corresponds to the background subtracted median fluorescence and Fmax was calculated from the plot.

Confocal imaging. Cancer cells were grown to about 80% confluency in 8-well confocal imaging chamber plates. Cells were incubated with 30 nM Tras-FITC for 30 min and then fixed by 4% paraformaldehyde for 15 min. Cells were washed three times with PBS (pH 7.4) and then incubated with DiIC18(3) (Marker Gene Technologies, Inc.) for 20 min and Hoechst 33342 (Cat No: H1399, Life Technologies™) for 5 min. Cells were then washed three times with PBS (pH 7.4) and used for confocal imaging. Confocal fluorescence images of cells were obtained using a Nikon AiR-si Laser Scanning Confocal Microscope (Japan), equipped with lasers of 405/488/561/638 nm.

Binding to bone cryosections. Nondecalcified long bone sections from C57BL/6 mice were incubated with 50 μg/mL Tras or Tras-ALN, conjugated overnight at 4° C., followed by staining with fluorescein isothiocyanate (FITC)-labeled anti-human IgG for 60 min at room temperature. After washing 3 times with PBS, specimens were incubated for 30 min at 37° C. with Xylenol Orange (XO) (stock: 2 mg/ml, dilute 1:500, dilute buffer: PBS pH 6.5). After three washes with PBS, specimens were stained with Hoechst 33342 (stock 10 mg/ml, dilute 1:2000) for 10 min. Slides were then washed with PBS, air dried, and sealed with Prolong™ gold anti-fade mountant (from ThermoFisher).

In vivo evaluation of Tras-ALN. Intra-iliac injections and IVIS imaging were performed as previously described.⁵³ Five days after injection, animals were randomized into four groups: PBS treated control, ALN (a representative of free BP, retro-orbital injection 10 g/kg in PBS twice a week), Tras (1.0 mg/kg retro-orbital injection in sterile PBS twice a week), and Tras-ALN conjugate (same as Tras). After injection, animals were imaged twice a week using IVIS Lumina II (Advanced Molecular Vision), following the recommended procedures and manufacturer's settings. On day 110, mice were anesthetized and blood was collected by cardiac puncture prior to euthanasia. Tumor-bearing tibia, heart, liver, spleen, lung, brain and kidney were collected for further tests.

Ex vivo metastasis-to-metastasis analysis. Mice were anesthetized with 2.5% isoflurane in oxygen and injected with luciferin retro-orbitally. Mice were then euthanized and their hearts, livers, spleens, lungs, kidneys, brain, and tibia bones were collected. Ex vivo bioluminescence and fluorescence imaging of these organs were immediately performed on the IVIS Lumina.

Bone histology and immunohistochemistry. Harvested long bones were fixed for 1 week in 10% formalin and then decalcified in 12% EDTA at 4° C. for two weeks. Specimens were embedded in paraffin using the standard procedure. From these blocks, 5 m sections were cut and collected on glass slides. Sections were dried in an oven overnight (37° C.) and then deparaffinized in xylene solution for 10 min. Hematoxylin and eosin (H&E) staining were performed via the conventional method. Immunohistochemistry analysis was performed on decalcified paraffin-embedded tissue sections using the HRP/DAB ABC IHC KIT (abcam) following the manufacture's protocol.

Radiographic analysis. Tibiae were dissected, fixed and scanned by microcomputed tomography (micro-CT, Skyscan 1272, Aartselaar, Belgium) at a resolution of 6.64 m/pixel. Raw images were reconstructed in NReconn and analyzed in CTan (SkyScan, Aartselaar, Belgium) using a region of interest (ROI). Bone parameters analyzed included trabecular thickness (Tb.Th), bone volume fraction (BV/TV), bone mineral density (BMD), and BS/BV (bone surface/bone volume ratio).

Biodistribution. MDA-MB-361 cells were introduced into female athymic nude mice (body weight=13-15 g) via intra-iliac injections. After three months, Cy7.5-labeled Tras and Tras-ALN (1 mg/kg) were administrated to tumor-bearing nude mice by retro-orbital injection. At 24 h, 96 h, or 168 h after injection, major organs including heart, liver, spleen, kidney, lung, and bone tumor tissue were removed. The fluorescence intensity in organs and bone tumor tissues was determined semiquantitatively by using the Caliper IVIS Lumina in vivo imager (Caliper Life Science, Boston, MA, USA). Bones from Tras-ALN treated mice were fixed and sectioned to further evaluate biodistribution.

In a separate study, unlabeled Tras-ALN (1 mg/kg) was administered via retro-orbital injection to nude mice bearing MDA-MB-361 tumors in their right hind limbs. After 48 hours, long bones from Tras-ALN treated mice were isolated and immediately sectioned without decalcification. Bone sections were then fixed and incubated with anti-RFP (rabbit) antibody (1:200, purchased from Rockland) overnight at 4, followed by staining with fluorescein isothiocyanate (FITC)-labeled anti-human IgG (1:100, purchased from Jackson Immunology) and Alexa Fluor® 555 AffiniPure Donkey Anti-Rabbit IgG (H+L) (1:200, purchased from Thermo Fisher) for 120 min at room temperature. Sections were mounted with Prolong™ gold anti-fade mountant with DAPI (from ThermoFisher) and sealed with a coverslip, then used for confocal imaging.

Pharmacokinetic Analysis and FcRn binding assay. Athymic nude mice were injected retro-orbitally with a single dose of 1 mg/kg Tras and Tras-ALN in PBS, and serum was collected at regular intervals for 7 d and analyzed by Trastuzumab ELISA Kit (Lab Bioreagents). FcRn binding was determined using LUMIT™ FcRn Binding Immunoassay kit (Promega) according to the manual.

Quantification of TRAP and calcium levels in serum. At terminal time points, blood was collected by cardiac puncture, and centrifuged for 15 min at 3,000 rpm to obtain the serum. The concentration of osteoclast-derived TRACP 5b was measured by using a Mouse ACP5/TRAP ELISA Kit (catalog number IT5180, GBiosciences). Serum calcium levels were determined colorimetrically using a calcium detection kit (catalog number DICA-500, Bioassays).

Statistical methods. Data are presented as means plus or minus SEM and statistically analyzed using GraphPad Prism software version 6 (GraphPad software, San Diego, CA). Two-way ANOVA followed by Sidak's multiple comparisons was used for all data collected over a time course. One-way ANOVA followed by Tukey's multiple comparisons was used for Micro-CT data. Unpaired Student's t-test was used for multi-organ metastasis data. P<0.05 was considered to represent statistical significance.

Synthesis of BCN-ALN. BCN-PNP (ENDO) (31.5 mg) and DIPEA (38.7 mg) were dissolved in 1 mL dimethylsulfoxide (DMSO), followed by dropwise addition of 27.4 mg of ALN (dissolved in 0.3 mL of deionized water) into the mixture. The resulting mixture was stirred for 4 hours. Ethyl acetate (1 mL) was added to the reaction solution, and the resulting precipitate was filtered and rinsed three times with ethyl acetate. The product was purified by reversed-phase column chromatography. The structure of BCN-ALN was confirmed by MS. ESI-MS [M−H]−: Calcd. For C15H25NO9P2 424.1, found: 424.1 (FIG. 5).

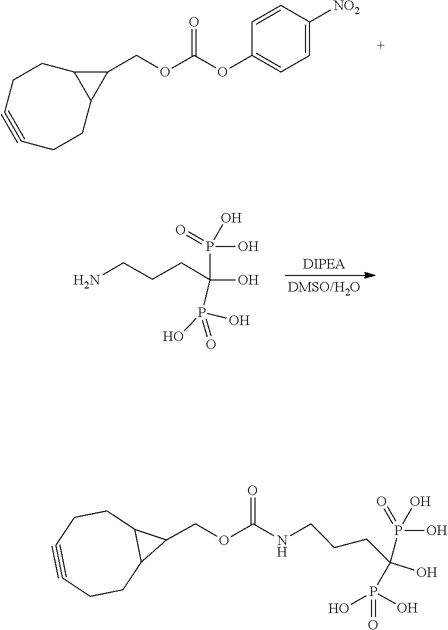 [SMILES: O=C(OCC1C2CCC#CCCC21)Oc1ccc([N+](=O)[O-])cc1 ; NCCCC(O)(P(=O)(O)O)P(=O)(O)O ; O=C(NCCCC(O)(P(=O)(O)O)P(=O)(O)O)OCC1C2CCC#CCCC21]

Solid-phase synthesis of ssFB-FPheK peptide. ssFB peptide was synthesized by following the protocol for Fmoc-based peptide synthesis. Amino acid sequence: FNKEQQNAFYEIHLPNLNXEQRNAFIQSLKDDPS-AzK (X=MMT-Lys) (SEQ ID NO: 1). Rink Amide MBHA resin was used as the solid support. The Fmoc protection group was removed using 25% piperidine in DMF. After washing the product five times with DMF, pre-activated HATU/Fmoc-amino acid mixture in DMF was added to the reaction vessel for amide bond formation. The next amino acid was then coupled to the beads via the same reaction cycle. Once peptide synthesis was complete, the N-terminus was capped by acetic anhydride. In order to incorporate FPheK into the peptide, the MMT protection group was first selectively removed using 10% acetic acid (AcOH:TFE:DCM=1:2:7). Subsequently, 2 equiv of 4-fluorophenyl chloroformate and 4 equiv of DIEA were added into the reaction vessel to react with the exposed free amine on the Lys side chain for FPheK formation. Once the reaction was complete, appropriate quantities of TFA and scavengers (water, anisole, triisopropyl silane) were added to the vessel to cleave the peptide from the resin, and to remove and quench all protection groups. The peptide was then precipitated by addition of ice-cold ether, purified by HPLC and lyophilized. ESI-MS [M+H]+: Calcd. For 4524, found: 4524 (FIG. 6).

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
(1) Scott, A. M.; Wolchok, J. D.; Old, L. J. Antibody Therapy of Cancer. Nat. Rev. Cancer 2012, 12 (4), 278-287. https://doi.org/10.1038/nrc3236.
(2) Sievers, E. L.; Senter, P. D. Antibody-Drug Conjugates in Cancer Therapy. Annu. Rev. Med. 2013, 64 (1), 15-29. https://doi.org/10.1146/annurev-med-050311-201823.
(3) Pardoll, D. M. The Blockade of Immune Checkpoints in Cancer Immunotherapy. Nat. Rev. Cancer 2012, 12 (4), 252-264. https://doi.org/10.1038/nrc3239.
(4) Labrijn, A. F.; Janmaat, M. L.; Reichert, J. M.; Parren, P. W. H. I. Bispecific Antibodies: A Mechanistic Review of the Pipeline. Nat. Rev. Drug Discov. 2019, 18 (8), 585-608. https://doi.org/10.1038/s41573-019-0028-1.
(5) Tolcher, A. W. Antibody Drug Conjugates: Lessons from 20 Years of Clinical Experience. Ann. Oncol. 2016, 27 (12), 2168-2172. https://doi.org/10.1093/annonc/mdw424.
(6) Hansel, T. T.; Kropshofer, H.; Singer, T.; Mitchell, J. A.; George, A. J. T. The Safety and Side Effects of Monoclonal Antibodies. Nat. Rev. Drug Discov. 2010, 9 (4), 325-338. https://doi.org/10.1038/nrd3003.
(7) Descotes, J. Immunotoxicity of Monoclonal Antibodies. mAbs 2009, 1 (2), 104-111.
(8) Baxter, L. T.; Zhu, H.; Mackensen, D. G.; Butler, W. F.; Jain, R. K. Biodistribution of Monoclonal Antibodies: Scale-up from Mouse to Human Using a Physiologically Based Pharmacokinetic Model. Cancer Res. 1995, 55 (20), 4611-4622.
(9) Coleman, R. E.; Rubens, R. D. The Clinical Course of Bone Metastases from Breast Cancer. Br. J. Cancer 1987, 55 (1), 61-66. https://doi.org/10.1038/bjc.1987.13.
(10) Coleman, R. E.; Smith, P.; Rubens, R. D. Clinical Course and Prognostic Factors Following Bone Recurrence from Breast Cancer. Br. J. Cancer 1998, 77 (2), 336-340. https://doi.org/10.1038/bjc.1998.52.
(11) Mundy, G. R. Metastasis to Bone: Causes, Consequences and Therapeutic Opportunities. Nat. Rev. Cancer 2002, 2 (8), 584-593. https://doi.org/10.1038/nrc867.
(12) Kingsley, L. A.; Fournier, P. G. J.; Chirgwin, J. M.; Guise, T. A. Molecular Biology of Bone Metastasis. Mol. Cancer Ther. 2007, 6 (10), 2609-2617. https://doi.org/10.1158/1535-7163.MCT-07-0234.
(13) Ell, B.; Kang, Y. SnapShot: Bone Metastasis. Cell 2012, 151 (3), 690-690.el. https://doi.org/10.1016/j.cell.2012.10.005.
(14) Fornetti, J.; Welm, A. L.; Stewart, S. A. Understanding the Bone in Cancer Metastasis. J. Bone Miner. Res. Off J. Am. Soc. Bone Miner. Res. 2018, 33 (12), 2099-2113. https://doi.org/10.1002/jbmr.3618.
(15) Brown, D.; Smeets, D.; Szekely, B.; Larsimont, D.; Szisz, A. M.; Adnet, P.-Y.; Rothe, F.; Rouas, G.; Nagy, Z. I.; Faragó, Z.; Töké]s, A.-M.; Dank, M.; Szentmartoni, G.; Udvarhelyi, N.; Zoppoli, G.; Pusztai, L.; Piccart, M.; Kulka, J.; Lambrechts, D.; Sotiriou, C.; Desmedt, C. Phylogenetic Analysis of Metastatic Progression in Breast Cancer Using Somatic Mutations and Copy Number Aberrations. Nat. Commun. 2017, 8, 14944. https://doi.org/10.1038/ncomms14944.
(16) Siegel, M. B.; He, X.; Hoadley, K. A.; Hoyle, A.; Pearce, J. B.; Garrett, A. L.; Kumar, S.; Moylan, V. J.; Brady, C. M.; Van Swearingen, A. E.; Marron, D.; Gupta, G. P.; Thorne, L. B.; Kieran, N.; Livasy, C.; Mardis, E. R.; Parker, J. S.; Chen, M.; Anders, C. K.; Carey, L. A.; Perou, C. M. Integrated RNA and DNA Sequencing Reveals Early Drivers of Metastatic Breast Cancer. J. Clin. Invest. 2018, 128 (4), 1371-1383. https://doi.org/10.1 172/JC196153.
(17) Ullah, I.; Karthik, G.-M.; Alkodsi, A.; Kjallquist, U.; Stilhammar, G.; Lovrot, J.; Martinez, N.-F.; Lagergren, J.; Hautaniemi, S.; Hartman, J.; Bergh, J. Evolutionary History of Metastatic Breast Cancer Reveals Minimal Seeding from Axillary Lymph Nodes. J. Clin. Invest. 2018, 128 (4), 1355-1370. https://doi.org/10.1172/JCI96149.
(18) Balic, M.; Lin, H.; Young, L.; Hawes, D.; Giuliano, A.; McNamara, G.; Datar, R. H.; Cote, R. J. Most Early Disseminated Cancer Cells Detected in Bone Marrow of Breast Cancer Patients Have a Putative Breast Cancer Stem Cell Phenotype. Clin. Cancer Res. Off J. Am. Assoc. Cancer Res. 2006, 12 (19), 5615-5621. https://doi.org/10.1158/1078-0432.CCR-06-0169.
(19) Marty, M.; Cognetti, F.; Maraninchi, D.; Snyder, R.; Mauriac, L.; Tubiana-Hulin, M.; Chan, S.; Grimes, D.; Antón, A.; Lluch, A.; Kennedy, J.; O'Byrne, K.; Conte, P.; Green, M.; Ward, C.; Mayne, K.; Extra, J.-M. Randomized Phase II Trial of the Efficacy and Safety of Trastuzumab Combined with Docetaxel in Patients with Human Epidermal Growth Factor Receptor 2-Positive Metastatic Breast Cancer Administered as First-Line Treatment: The M77001 Study Group. J. Clin. Oncol. Off J. Am. Soc. Clin. Oncol. 2005, 23 (19), 4265-4274. https://doi.org/10.1200/JCO.2005.04.173.
(20) Valero, V.; Forbes, J.; Pegram, M. D.; Pienkowski, T.; Eiermann, W.; von Minckwitz, G.; Roche, H.; Martin, M.; Crown, J.; Mackey, J. R.; Fumoleau, P.; Rolski, J.; Mrsic-Krmpotic, Z.; Jagiello-Gruszfeld, A.; Riva, A.; Buyse, M.; Taupin, H.; Sauter, G.; Press, M. F.; Slamon, D. J. Multicenter Phase III Randomized Trial Comparing Docetaxel and Trastuzumab with Docetaxel, Carboplatin, and Trastuzumab as First-Line Chemotherapy for Patients with HER2-Gene-Amplified Metastatic Breast Cancer (BCIRG 007 Study): Two Highly Active Therapeutic Regimens. J. Clin. Oncol. Off J. Am. Soc. Clin. Oncol. 2011, 29 (2), 149-156. https://doi.org/10.1200/JCO.2010.28.6450.
(21) Nahta, R.; Yu, D.; Hung, M.-C.; Hortobagyi, G. N.; Esteva, F. J. Mechanisms of Disease: Understanding Resistance to HER2-Targeted Therapy in Human Breast Cancer. Nat. Clin. Pract. Oncol. 2006, 3 (5), 269-280. https://doi.org/10.1038/ncponc0509.
(22) Gullo, G.; Zuradelli, M.; Sclafani, F.; Santoro, A.; Crown, J. Durable Complete Response Following Chemotherapy and Trastuzumab for Metastatic HER2-Positive Breast Cancer. Ann. Oncol. Off J. Eur. Soc. Med. Oncol. 2012, 23 (8), 2204-2205. https://doi.org/10.1093/annonc/mds221.
(23) Schmid, P.; Adams, S.; Rugo, H. S.; Schneeweiss, A.; Barrios, C. H.; Iwata, H.; Dieras, V.; Hegg, R.; Im, S.-A.; Shaw Wright, G.; Henschel, V.; Molinero, L.; Chui, S. Y.; Funke, R.; Husain, A.; Winer, E. P.; Loi, S.; Emens, L. A. Atezolizumab and Nab-Paclitaxel in Advanced Triple-Negative Breast Cancer. N. Engl. J. Med. 2018, 379 (22),
2108-2121. https://doi.org/10.1056/NEJMoa1809615.
(24) Cole, L. E.; Vargo-Gogola, T.; Roeder, R. K. Targeted Delivery to Bone and Mineral Deposits Using Bisphosphonate Ligands. Adv. DrugDeliv. Rev. 2016, 99 (Pt A), 12-27. https://doi.org/10.1016/j.addr.2015.10.005.
(25) Dang, L.; Liu, J.; Li, F.; Wang, L.; Li, D.; Guo, B.; He, X.; Jiang, F.; Liang, C.; Liu, B.; Badshah, S. A.; He, B.; Lu, J.; Lu, C.; Lu, A.; Zhang, G. Targeted Delivery Systems for Molecular Therapy in Skeletal Disorders. Int. J. Mol. Sci. 2016, 17 (3). https://doi.org/10.3390/ijms17030428.
(26) Stapleton, M.; Sawamoto, K.; Almeciga-Diaz, C. J.; Mackenzie, W. G.; Mason, R. W.; Orii, T.; Tomatsu, S. Development of Bone Targeting Drugs. Int. J. Mol. Sci. 2017, 18 (7). https://doi.org/10.3390/ijms18071345.
(27) Singh, T.; Kaur, V.; Kumar, M.; Kaur, P.; Murthy, R. S. R.; Rawal, R. K. The Critical Role of Bisphosphonates to Target Bone Cancer Metastasis: An Overview. J. Drug Target. 2015, 23 (1), 1-15. https://doi.org/10.3109/1061186X.2014.950668.
(28) Ebetino, F. H.; Hogan, A.-M. L.; Sun, S.; Tsoumpra, M. K.; Duan, X.; Triffitt, J. T.; Kwaasi, A. A.; Dunford, J. E.; Barnett, B. L.; Oppermann, U.; Lundy, M. W.; Boyde, A.; Kashemirov, B. A.; McKenna, C. E.; Russell, R. G. G. The Relationship between the Chemistry and Biological Activity of the Bisphosphonates. Bone 2011, 49 (1), 20-33. https://doi.org/10.1016/j.bone.2011.03.774.
(29) Farrell, K. B.; Karpeisky, A.; Thamm, D. H.; Zinnen, S. Bisphosphonate Conjugation for Bone Specific Drug Targeting. Bone Rep. 2018, 9, 47-60. https://doi.org/10.1016/j.bonr.2018.06.007.
(30) Young, R. N.; Grynpas, M. D. Targeting Therapeutics to Bone by Conjugation with Bisphosphonates. Curr. Opin. Pharmacol. 2018, 40, 87-94. https://doi.org/10.1016/j.coph.2018.03.010.
(31) Fleisch, H. Bisphosphonates in Bone Disease: From the Laboratory to the Patient; Elsevier, 2000.
(32) Bayouth, J. E.; Macey, D. J.; Kasi, L. P.; Fossella, F. V. Dosimetry and Toxicity of Samarium-153-EDTMP Administered for Bone Pain Due to Skeletal Metastases. J. Nucl. Med Off Publ. Soc. Nucl. Med 1994, 35 (1), 63-69.
(33) Hirabayashi, H.; Sawamoto, T.; Fujisaki, J.; Tokunaga, Y.; Kimura, S.; Hata, T. Relationship between Physicochemical and Osteotropic Properties of Bisphosphonic Derivatives: Rational Design for Osteotropic Drug Delivery System (ODDS). Pharm. Res. 2001, 18 (5), 646-651. https://doi.org/10.1023/a:1011033326980.
(34) Hochdorffer, K.; Abu Ajaj, K.; Schafer-Obodozie, C.; Kratz, F. Development of Novel Bisphosphonate Prodrugs of Doxorubicin for Targeting Bone Metastases That Are Cleaved PH Dependently or by Cathepsin B: Synthesis, Cleavage Properties, and Binding Properties to Hydroxyapatite as Well as Bone Matrix. J. Med Chem. 2012, 55 (17), 7502-7515. https://doi.org/10.1021/jm300493m.
(35) Goeckeler, W. F.; Edwards, B.; Volkert, W. A.; Holmes, R. A.; Simon, J.; Wilson, D. Skeletal Localization of Samarium-153 Chelates: Potential Therapeutic Bone Agents. J. Nucl. Med Off Publ. Soc. Nucl. Med 1987, 28 (4), 495-504.
(36) Yu, C.; Wang, H.; Muscarella, A.; Goldstein, A.; Zeng, H.-C.; Bae, Y.; Lee, B. H. I.; Zhang, X. H.-F. Intra-Iliac Artery Injection for Efficient and Selective Modeling of Microscopic Bone Metastasis. J. Vis. Exp. JoVE 2016, No. 115. https://doi.org/10.3791/53982.
(37) Wang, H.; Yu, C.; Gao, X.; Welte, T.; Muscarella, A. M.; Tian, L.; Zhao, H.; Zhao, Z.; Du, S.; Tao, J.; Lee, B.; Westbrook, T. F.; Wong, S. T. C.; Jin, X.; Rosen, J. M.; Osborne, C. K.; Zhang, X. H.-F. The Osteogenic Niche Promotes Early-Stage Bone Colonization of Disseminated Breast Cancer Cells. Cancer Cell 2015, 27 (2), 193-210. https://doi.org/10.1016/j.ccell.2014.11.017.
(38) Wang, H.; Tian, L.; Liu, J.; Goldstein, A.; Bado, I.; Zhang, W.; Arenkiel, B. R.; Li, Z.; Yang, M.; Du, S.; Zhao, H.; Rowley, D. R.; Wong, S. T. C.; Gugala, Z.; Zhang, X. H.-F. The Osteogenic Niche Is a Calcium Reservoir of Bone Micrometastases and Confers Unexpected Therapeutic Vulnerability. Cancer Cell 2018, 34 (5), 823-839.e7. https://doi.org/10.1016/j.ccell.2018.10.002.
(39) Hochdbrffer, K.; Abu Ajaj, K.; Schafer-Obodozie, C.; Kratz, F. Development of Novel Bisphosphonate Prodrugs of Doxorubicin for Targeting Bone Metastases That Are Cleaved PH Dependently or by Cathepsin B: Synthesis, Cleavage Properties, and Binding Properties to Hydroxyapatite As Well As Bone Matrix. J. Med. Chem. 2012, 55
(17), 7502-7515. https://doi.org/10.1021/jm300493m. (40) Magbanua, M. J. M.; Rugo, H. S.; Hauranieh, L.; Roy, R.; Scott, J. H.; Lee, J. C.; Hsiao, F.; Sosa, E. V.; van′t Veer, L.; Esserman, L. J.; Park, J. W. Genomic and Expression Profiling Reveal Molecular Heterogeneity of Disseminated Tumor Cells in Bone Marrow of Early Breast Cancer. Npj Breast Cancer 2018, 4 (1), 31. https://doi.org/10.1038/s41523-018-0083-5.
(41) Solomayer, E. F.; Becker, S.; Pergola-Becker, G.; Bachmann, R.; Kramer, B.; Vogel, U.; Neubauer, H.; Wallwiener, D.; Huober, J.; Fehm, T. N. Comparison of HER2 Status between Primary Tumor and Disseminated Tumor Cells In-primary Breast Cancer Patients. Breast Cancer Res. Treat. 2006, 98 (2), 179-184. https://doi.org/10.1007/s10549-005-9147-y.
(42) Ithimakin, S.; Day, K. C.; Malik, F.; Zen, Q.; Dawsey, S. J.; Bersano-Begey, T. F.; Quraishi, A. A.; Ignatoski, K. W.; Daignault, S.; Davis, A.; Hall, C. L.; Palanisamy, N.; Heath, A. N.; Tawakkol, N.; Luther, T. K.; Clouthier, S. G.; Chadwick, W. A.; Day, M. L.; Kleer, C. G.; Thomas, D. G.; Hayes, D. F.; Korkaya, H.; Wicha, M. S. HER2 Drives Luminal Breast Cancer Stem Cells in the Absence of HER2 Amplification: Implications for Efficacy of Adjuvant Trastuzumab. Cancer Res. 2013, 73 (5), 1635-1646. https://doi.org/10.1158/0008-5472.CAN-12-3349.
(43) Kennecke, H.; Yerushalmi, R.; Woods, R.; Cheang, M. C. U.; Voduc, D.; Speers, C. H.; Nielsen, T. O.; Gelmon, K. Metastatic Behavior of Breast Cancer Subtypes. J. Clin. Oncol. Off J. Am. Soc. Clin. Oncol. 2010, 28 (20), 3271-3277. https://doi.org/10.1200/JCO.2009.25.9820.
(44) Manders, K.; van de Poll-Franse, L. V.; Creemers, G.-J.; Vreugdenhil, G.; van der Sangen, M. J.; Nieuwenhuijzen, G. A.; Roumen, R. M.; Voogd, A. C. Clinical Management of Women with Metastatic Breast Cancer: A Descriptive Study According to Age Group. BMC Cancer 2006, 6 (1), 179. https://doi.org/10.1186/1471-2407-6-179.
(45) Kuchuk, I.; Hutton, B.; Moretto, P.; Ng, T.; Addison, C. L.; Clemons, M. Incidence, Consequences and Treatment of Bone Metastases in Breast Cancer Patients-Experience from a Single Cancer Centre. J. Bone Oncol. 2013, 2 (4), 137-144. https://doi.org/10.1016/j.jbo.2013.09.001.
(46) Svensson, E.; Christiansen, C. F.; Ulrichsen, S. P.; Rorth, M. R.; Sorensen, H. T. Survival after Bone Metastasis by Primary Cancer Type: A Danish Population-Based Cohort Study. BMJ Open 2017, 7 (9), e016022. https://doi.org/10.1136/bmjopen-2017-016022.
(47) Parkes, A.; Warneke, C. L.; Clifton, K.; Al-Awadhi, A.; Oke, O.; Pestana, R. C.; Alhalabi, O.; Litton, J. K.; Hortobagyi, G. N. Prognostic Factors in Patients with Metastatic Breast Cancer with Bone-Only Metastases. The Oncologist 2018, 23 (11), 1282-1288. https://doi.org/10.1634/theoncologist.2018-0085.
(48) Magbanua, M. J. M.; Rugo, H. S.; Hauranieh, L.; Roy, R.; Scott, J. H.; Lee, J. C.; Hsiao, F.; Sosa, E. V.; Van′t Veer, L.; Esserman, L. J.; Park, J. W. Genomic and Expression Profiling Reveal Molecular Heterogeneity of Disseminated Tumor Cells in Bone Marrow of Early Breast Cancer. NPJ Breast Cancer 2018, 4, 31. https://doi.org/10.1038/s41523-018-0083-5.
(49) Solomayer, E. F.; Becker, S.; Pergola-Becker, G.; Bachmann, R.; Kramer, B.; Vogel, U.; Neubauer, H.; Wallwiener, D.; Huober, J.; Fehm, T. N. Comparison of HER2 Status between Primary Tumor and Disseminated Tumor Cells in Primary Breast Cancer Patients. Breast Cancer Res. Treat. 2006, 98 (2), 179-184. https://doi.org/10.1007/s10549-005-9147-y.
(50) Kingsley, L. A.; Fournier, P. G. J.; Chirgwin, J. M.; Guise, T. A. Molecular Biology of Bone Metastasis. Mol. Cancer Ther. 2007, 6 (10), 2609-2617. https://doi.org/10.1158/1535-7163.MCT-07-0234.
(51) Wang, H.; Tian, L.; Goldstein, A.; Liu, J.; Lo, H.-C.; Sheng, K.; Welte, T.; Wong, S. T. C.; Gugala, Z.; Stossi, F.; Zong, C.; Li, Z.; Mancini, M. A.; Zhang, X. H.-F. Bone-in-Culture Array as a Platform to Model Early-Stage Bone Metastases and Discover Anti-Metastasis Therapies. Nat. Commun. 2017, 8 (1), 15045. https://doi.org/10.1038/ncomms15045.
(52) Dillon, R. L.; Chooniedass, S.; Premsukh, A.; Adams, G. P.; Entwistle, J.; MacDonald, G. C.; Cizeau, J. Trastuzumab-DeBouganin Conjugate Overcomes Multiple Mechanisms of T-DM1 Drug Resistance: J. Immunother. 2016, 39 (3), 117-126. https://doi.org/10.1097/CJI.0000000000000115.
(53) Wang, H.; Yu, C.; Gao, X.; Welte, T.; Muscarella, A. M.; Tian, L.; Zhao, H.; Zhao, Z.; Du, S.; Tao, J.; Lee, B.; Westbrook, T. F.; Wong, S. T. C.; Jin, X.; Rosen, J. M.; Osborne, C. K.; Zhang, X. H.-F. The Osteogenic Niche Promotes Early-Stage Bone Colonization of Disseminated Breast Cancer Cells. Cancer Cell 2015, 27 (2), 193-210. https://doi.org/10.1016/j.ccell.2014.11.017.
Junutula et al., Site-Specific Conjugation of a Cytotoxic Drug to an Antibody Improves the Therapeutic Index. Nat. Biotechnol. 26 (8), 925-932, 2008.
Carrico et al., SMARTag, which genetically encodes a peptide tag for further enzymatic modification; (2007) Introducing Genetically Encoded Aldehydes into Proteins. Nat. Chem. Biol. 3 (6), 321-322.
Boeggeman et al., SiteClick labeling system, which introduces an unnatural sugar; (2009) Site Specific Conjugation of Fluoroprobes to the Remodeled Fc NGlycans of Monoclonal Antibodies Using Mutant Glycosyltransferases: Application for Cell Surface Antigen Detection. Bioconjugate Chem. 20 (6), 1228-1236
Leisle et al., Noncanonical amino acid (ncAA) technology that enables site-specific incorporation of the 21st amino acid with a distinct reactive moiety; (2012) Synthesis of Site-Specific Antibody-Drug Conjugates Using Unnatural Amino Acids. Proc. Natl. Acad. Sci. U.S.A 109 (40), 16101-16106.
Zhang et al., Disulfide bridging, selectively cleave the disulfide bond first, and subsequently reconnect two cysteines together by the introduction of a short covalent bridge between them; Bioconjugate Chem., 2014, 25, 1124-1136
LLQC tag; the tag can be recognized by mTG for transferring amine containing drug; Chem. Biol. 2013, 20, 161-167.

1. A method of treating or preventing bone tumors in a subject comprising administering to the subject an effective amount of a bone-targeting conjugate comprising bisphosphonate (BP) conjugated to an antibody.

2. The method of claim 1, wherein the subject has bone cancer or bone metastasis.

3. The method of claim 2, wherein the bone cancer is Ewing sarcoma, osteosarcoma, or chondrosarcoma.

4. The method of claim 2, wherein the bone metastasis is from breast cancer, myeloma, renal cancer, lung cancer, prostate cancer, thyroid cancer, or bladder cancer.

5. The method of claim 1, wherein the BP is negatively-charged.

6. The method of claim 1, wherein the BP is alendronate, zoledronate, pamidronate, risedronate, medronic acid, aminomethylene bisphonic acid, clodronate, etidronate, tiludronate, ibandronate pomidronate, neridonate, olpadronate, or oxidronate.

7. The method of claim 1, wherein the BP is alendronate (ALN).

8. The method of claim 4, wherein the breast cancer is triple-negative breast cancer.

9. The method of claim 4, wherein the breast cancer is HER2-negative breast cancer.

10. The method of claim 4, wherein the breast cancer is HER2-positive breast cancer.

11. The method of claim 1, wherein the antibody is a monoclonal antibody, bispecific antibody, Fab′, a F(ab′)2, a F(ab′)3, a monovalent scFv, a bivalent scFv, a single domain antibody, or nanobody.

12. The method of claim 1, wherein the antibody is an immune checkpoint inhibitor.

13. The method of claim 1, wherein the antibody is an anti-HER2 antibody, anti-CD99 antibody, anti-IGF-IR antibody, anti-PD-L1, anti-PD-1, anti-CTLA4-antibody, anti-Siglec-15 antibody, anti-RANKL antibody, or anti-TGFβ antibody.

14. The method of claim 1, wherein the antibody is an anti-HER2 antibody.

15. The method of claim 1, wherein the antibody is trastuzumab (Herceptin), pertuzumab (Perjeta), or atezolizumab.

16. The method of claim 1, wherein the antibody is trastuzumab.

17. The method of claim 1, wherein bone-targeting conjugate comprises alendronate conjugated to trastuzumab.

18. The method of claim 1, wherein the antibody is not an anti-M-CSF antibody.

19. The method of claim 1, wherein the BP is not conjugated to N-glycan on the Fc region of the antibody.

20. The method of claim 1, wherein the BP is site-specifically conjugated to the antibody using pClick conjugation, NHS-ester chemistry, or cysteine chemistry.

21. The method of claim 1, wherein the BP is site-specifically conjugated to the antibody using pClick conjugation.

22. The method of claim 1, wherein the BP is conjugated to the CH2-CH3 junction of the antibody.

23. The method of claim 1, wherein the BP is conjugated to the antibody using 4-fluorophenyl carbamate lysine (FPheK).

24. The method of claim 23, wherein FPheK is attached to a fragment of the B domain of protein A (FB protein) from Staphylococcus aureus.

25. The method of claim 21, wherein pClick conjugation comprises conjugation of an antibody with an azide functional moiety with BP functionalized with bicyclo[6.1.0]nonyne (BCN).

26. The method of claim 1, wherein the bone-targeting conjugate results in increased concentration of therapeutic antibody at the bone tumor niche, inhibits cancer development in the bone, and/or limits secondary metastases to other organs.

27. The method of claim 1, wherein the bone-targeting conjugate results in decreased micrometastasis-induced osteolyic lesions.

28. The method of claim 1, wherein the method comprises further administering an additional anti-cancer therapy.

29. The method of claim 28, wherein the additional anti-cancer therapy comprises surgery, chemotherapy, radiation therapy, hormonal therapy, immunotherapy or cytokine therapy.

30. The method of claim 28, wherein the additional anti-cancer therapy comprises immunotherapy or chemotherapy.

31-41. (canceled)

42. A method of treating or preventing bone disease in a subject comprising administering to the subject an effective amount of a bone-targeting conjugate comprising bisphosphonate (BP) conjugated to one or more polypeptides.

43. The method of claim 42, wherein the bone disease is osteoporosis, osteomalacia, periodontitis, rheumatoid arthritis, metabolic bone disease, a parathyroid disorder, steroid-induced osteoporosis, chemotherapy-induced bone loss, pre-menopausal bone loss, fragility and recurrent fractures, renal osteodystrophy, bone infections, or Paget's disease.

44. The method of claim 42, wherein the bone disease is bone cancer or bone metastasis.

45. The method of claim 44, wherein the bone cancer is Ewing sarcoma, osteosarcoma, or chondrosarcoma.

46. The method of claim 44, wherein the bone metastasis is from breast cancer, myeloma, renal cancer, lung cancer, prostate cancer, thyroid cancer, or bladder cancer.

47. The method of claim 42, wherein the BP is negatively-charged.

48. The method of claim 42, wherein the BP is alendronate, zoledronate, pamidronate, risedronate, medronic acid, aminomethylene bisphonic acid, clodronate, etidronate, tiludronate, ibandronate pomidronate, neridonate, olpadronate, or oxidronate.

49. The method of claim 42, wherein the BP is alendronate (ALN).

50. The method of claim 46, wherein the breast cancer is triple-negative breast cancer.

51. The method of claim 46, wherein the breast cancer is HER2-negative breast cancer.

52. The method of claim 46, wherein the breast cancer is HER2-positive breast cancer.

53. The method of claim 42, wherein the one or more polypeptides comprise an adrenergic agonist, an anti-apoptosis factor, an apoptosis inhibitor, a cytokine receptor, a cytokine, a cytotoxin, an erythropoietic agent, a glutamic acid decarboxylase, a glycoprotein, a growth factor, a growth factor receptor, a hormone, a hormone receptor, an interferon, an interleukin, an interleukin receptor, a kinase, a kinase inhibitor, a nerve growth factor, a netrin, a neuroactive peptide, a neuroactive peptide receptor, a neurogenic factor, a neurogenic factor receptor, a neuropilin, a neurotrophic factor, a neurotrophin, a neurotrophin receptor, an N-methyl-D-aspartate antagonist, a plexin, a protease, a protease inhibitor, a protein decarboxylase, a protein kinase, a protein kinase inhibitor, a proteolytic protein, a proteolytic protein inhibitor, a semaphorin, a semaphorin receptor, a serotonin transport protein, a serotonin uptake inhibitor, a serotonin receptor, a serpin, a serpin receptor, or a tumor suppressor.

54. The method of claim 45, wherein the one or more polypeptides comprise an antibody.

55. The method of claim 54, wherein the antibody is a monoclonal antibody, bispecific antibody, Fab′, a F(ab′)2, a F(ab′)3, a monovalent scFv, a bivalent scFv, a single domain antibody, or nanobody.

56. The method of claim 54, wherein the antibody is an immune checkpoint inhibitor.

57. The method of claim 54, wherein the antibody is an anti-HER2 antibody, anti-CD99 antibody, anti-IGF-IR antibody, anti-PD-L1, anti-PD-1, anti-CTLA4-antibody, anti-Siglec-15 antibody, anti-RANKL antibody, or anti-TGFβ antibody.

58. The method of claim 54, wherein the antibody is an anti-HER2 antibody.

59. The method of claim 54, wherein the antibody is trastuzumab (Herceptin), pertuzumab (Perjeta), or atezolizumab.

60. The method of claim 54, wherein the antibody is trastuzumab.

61. The method of claim 42, wherein bone-targeting conjugate comprises alendronate conjugated to trastuzumab.

62. The method of claim 54, wherein the antibody is not an anti-M-CSF antibody.

63. The method of claim 54, wherein the BP is not conjugated to N-glycan on the Fc region of the antibody.

64. The method of claim 42, wherein the BP is site-specifically conjugated to the one or more polypeptides using pClick conjugation, NHS-ester chemistry, or cysteine chemistry.

65. The method of claim 42, wherein the BP is site-specifically conjugated to the one or more polypeptides using pClick conjugation.

66. The method of claim 54, wherein the BP is conjugated to the CH2-CH3 junction of the antibody.

67. The method of claim 42, wherein the BP is conjugated to the one or more polypeptides using 4-fluorophenyl carbamate lysine (FPheK).

68. The method of claim 67, wherein FPheK is attached to a fragment of the B domain of protein A (FB protein) from Staphylococcus aureus.

69. The method of claim 65, wherein pClick conjugation comprises conjugation of an antibody with an azide functional moiety with BP functionalized with bicyclo[6.1.0]nonyne (BCN).

70. The method of claim 54, wherein the bone-targeting conjugate results in increased concentration of therapeutic antibody at the bone tumor niche, inhibits cancer development in the bone, and/or limits secondary metastases to other organs.

71. The method of claim 44, wherein the bone-targeting conjugate results in decreased micrometastasis-induced osteolyic lesions.

72. The method of claim 44, wherein the method comprises further administering an additional anti-cancer therapy.

73. The method of claim 72, wherein the additional anti-cancer therapy comprises surgery, chemotherapy, radiation therapy, hormonal therapy, immunotherapy or cytokine therapy.

74. The method of claim 72, wherein the additional anti-cancer therapy comprises immunotherapy or chemotherapy.

75. (canceled)
